SEQUENCE LISTING (43 sequences)

SEQ ID NO: 1 - DNA, 1560 bp, Francisella tularensis
cgttattcccattcaattgttccaggtggttttcctgtcacatcatacacaactcttgatacttgttttacttcatttacaattctatttgaaactagtgacagaaaatcataaggtaaattagcccacgttgcagtcataaaatcgatactaacgacagctcttagagctattacatactcatatctacgctgatcaccaactacacctaccgatttgacaggtaaaaacaccccaaaagcttgagaaacatcatggtataaattatgtttatagagctcttctgtaaatatagcatcagctttttgtaacgtttctacatactctttctttatttcacctaatatacgtactccaagaccaggaccaggaaatggatgtctataaagcatattataaggtaatcctaaacctaagcctaatttacgaacctcatctttgaaaagctctcttaatggttctaaaagcttaagtttcatttcttttggtaagccaccaacattatgatgtgatttaattacatgcgctttagactgattattgccagctgattcaattacatcactgtagatagtgccctgcgcaagccatttagcattctctatcttagcagcctcttcatcaaaaatatcaacaaatagcttaccgataattttacgtttttgttctggatcacagataccccttagggcatctaagaatctattttttgcattgatacgaataacattaatatccatatgctctgcaaatacttgcataacttgatcaccctcattaaggcgcagtaaaccagtatctacaaatatacaagttagctgatcgccgatagcttgatgtaatatcgcagctacaacagatgaatcaacacctccagatagccctaaaattactttatcagtaccaactttttgtttaatttcttttatatcattttcgataatgttttcgatattccacagagtatcacatccgcagatattaacgacaaaattctcaataatttgcttaccattttctgtatgagtagtttctgggtgaaactgcacaccaaaaaaaggcttatttttatgtgcaactgccgcaactggagcatttgttgaagaagcaattatctcaaaatgctcaccagtttgagtgactttgtcactatgactcatccaaactagctgctcatgctccatatttgaaaatatgttatttgttgaatttaggatatttataattgccttaccaaattcactttgatcagctccttttacttcgccaccatgctgcattaccattgtttgcatgccataacaaatacccaaaacaggtactccaagctcaaaaactatctcaggagctttgacatcagaatcataaactgactcaggaccacctgataaaattattccttttgcttgataatttttgataaaatcagctgctacatcatgagggaaaatttcacaaaaaacacctacttctcttactctacgagcaataagttgtgtatattgcgagccaaaatctaaaatcaaaatcttatgattatgtatatctgtcattttttat

SEQ ID NO: 2 - DNA, 2040 bp, Francisella tularensis
tatattttctgcatatccaaaaggcataacagaaaacatcttaccaacaacaccgcttacagcaaactttttaggtataaagaagtatcttgaataaatatctataaactcaatctctatcttttgcttatagtttttagcaatactaatattgcataaaaaatgatccatctcaccctcagaagcgccaaatacaaaaacctttaaagccccaagagagattaaataattcaaagatttctcaaaatcagtcgaatattgatcagtatcaattaaaaacaaatcatcttctgataaagtaaatgaatcaaaatctccaattactttttttatttttgaatttaaatgtaatgattctttaactttttgataagccccatcagcacaaaatatatcaagttttgcaaaattatcttcgatatatttctcacaaaaacacaaatctactttaccatttagaaaaagtatcgcttcagacattgagttattaaatattaagtaaaatttaatagattataacttattaaaatcataattaagaggttttaagactggtaattaggtggttctttagtaatcgtgacgttatgtacatgcgattcattaaatccagcacctgtaatttgtacaaatgtcggttctgttcgcattgtttggatatcttttgagccagtatagcccatactagatttcagaccacctattagctgatgaatcactgcagataataaacctttgtatggtactctgccttcaacgccttctggaacaaactttttcgcctctgtttcactttggaaatatctatcagatgagcctttctccatagcaccaagagaacccataccacgatatgatttataagagcgaccttggaaaagctcaacctcccctggtgattcttcagtacctgcaaaaagccctccaatcataactacagatgctccagcaactatagcttttgcaatatcaccggaatatctaataccgccatctgcaatcacaggcactccagttccttttaatgcctctgcaacattagctatcgctgtaatttgtggtacaccaacaccagcaacaattctggtagtacagattgagccaggtccaataccaaccttgacagcatcagcaccagctttgactagatctttagcagcttctgctgtagcgatattaccaccgataacctgaatatgtggatagttctctttaacccacttaactgtatctaacacaccttgtgaatgtccatgagcagtatctacaacaataatatcgactccttcagctgccaatgctgcaacacgctctttagtattagcagcggttccaacagcagcgccaactctcaaacgcccaagagaatctttacaagcatttggtttattttgtgatctttcgatatcttttgttgtaataagaccgaccaactcgccttgttcattaactacaagcaacttctcaattttatgctcatgaagtttctttttaatcgctccttgagaagcatcttcaggcactgttacaagtttttctctaggtgtcataatagagctaacaggttcatctaaatcttttgcaaatctaaaatcacgccttgtaacaatacctatgattttattattatcatcaacaacgggaaaaccagagaaattatgttctttagctagttgcatgatttctttaattgagctttcttgtttaatggtaatgggatcaatcaccattccattttcaaatctttttacctttttaacttcttgggcttgggcttgaatagacatattcttatgaataataccaataccaccttcctgggctatcgcaatagctaggcgtgactcagtaaccgtatccatagctgcagatactaaaggaatatttaactgaatatctctagttatatttgtcttcaaatcaacttgatgaggaagaacatttgaatatctaggtgatagcaatacatcatcaaaagtgattgcctgctgagtaattcttaacattttttgatctccgtaattaaaatctaaagagtat

SEQ ID NO: 3 - DNA, 38 bp, Artificial Sequence
  chemically synthesized PCT primer
atccccggggatatcgtaagttggcagcatcacccgac

SEQ ID NO: 4 - DNA, 36 bp, Artificial Sequence
  chemically synthesized PCR primer
gcccatctctagaaactggcctcaggcatttgagaa

SEQ ID NO: 5 - DNA, 39 bp, Artificial Sequence
  chemically synthesized PCR primer
cccggatccatctttagagataagttttcacatattgag

SEQ ID NO: 6 - DNA, 50 bp, Artificial Sequence
  chemically synthesized PCR primer
cccgtcgacgcggccgcagatctataccgccagcatgatttaaggcttca

SEQ ID NO: 7 - DNA, 39 bp, Artificial Sequence
  chemically synthesized PCR primer
cccggatcctaaatataagcctaaggcagtgattgattt

SEQ ID NO: 8 - DNA, 45 bp, Artificial Sequence
  chemically synthesized PCR primer
cccgtcgacgcggccgcaacaacaccgcttacagcaaacttttta

SEQ ID NO: 9 - DNA, 43 bp, Artificial Sequence
  chemically synthesized PCR primer
gatgatatcgctatgtcaaaatatactattttagataaaataa

SEQ ID NO: 10 - DNA, 46 bp, Artificial Sequence
  chemically synthesized PCR primer
gatgatatctttttataaaaataaacattttcttaaaaggttattt

SEQ ID NO: 11 - DNA, 36 bp, Artificial Sequence
  chemically synthesized PCR primer
atgcccggggaagttcctatactttctagagaatag

SEQ ID NO: 12 - DNA, 35 bp, Artificial Sequence
  chemically synthesized PCR primer
atgcccgggagttcctattccgaagttcctattct

SEQ ID NO: 13 - DNA, 57 bp, Artificial Sequence
  chemically synthesized PCR primer
gatccccgaattcctgcagcccgggtttttgatctccgtaattaaaatctaaagagt

SEQ ID NO: 14 - DNA, 48 bp, Artificial Sequence
  chemically synthesized PCR primer
cccgaattcctgcagaacctcttaattatgattttaataagttataat

SEQ ID NO: 15 - DNA, 29 bp, Artificial Sequence
  chemically synthesized PCR primer
ggataaaataaccttttaagaaaatgttt

SEQ ID NO: 16 - DNA, 27 bp, Artificial Sequence
  chemically synthesized PCR primer
actctttagattttaattacggagatc

SEQ ID NO: 17 - DNA, 22 bp, Artificial Sequence
  chemically synthesized PCR primer
atgcagccgccgcattgcatca

SEQ ID NO: 18 - DNA, 25 bp, Artificial Sequence
  chemically synthesized PCR primer
tctttttaatcgctccttgagaagc

SEQ ID NO: 19 - DNA, 23 bp, Artificial Sequence
  chemically synthesized PCR primer
gtttctgggtgaaactgcacacc

SEQ ID NO: 20 - DNA, 44 bp, Artificial Sequence
  chemically synthesized PCR primer
gaattccggatcctttcttgaaaattttttttttgactcaatat

SEQ ID NO: 21 - DNA, 43 bp, Artificial Sequence
  chemically synthesized PCR primer
gaattcccccgggcttactcctttgttaaattatttttgttta

SEQ ID NO: 22 - DNA, 40 bp, Artificial Sequence
  chemically synthesized PCR primer
gaattcccccgggatgaacatcaaaaagtttgcaaaacaa

SEQ ID NO: 23 - DNA, 41 bp, Artificial Sequence
  chemically synthesized PCR primer
ctagctaggatccgatatcaagctggggtgggcgaagaact

SEQ ID NO: 24 - DNA, 30 bp, Artificial Sequence
  chemically synthesized PCR primer
gctagataggtgcctcactgattaagcatt

SEQ ID NO: 25 - DNA, 36 bp, Artificial Sequence
  chemically synthesized PCR primer
atcgctaggatccgcgaaagggggatgtgctgcaag

SEQ ID NO: 26 - DNA, 30 bp, Artificial Sequence
  chemically synthesized PCR primer
taaatataagcctaaggcagtgattgattt

SEQ ID NO: 27 - DNA, 31 bp, Artificial Sequence
  chemically synthesized PCR primer
ttttgatctccgtaattaaaatctaaagagt

SEQ ID NO: 28 - DNA, 30 bp, Artificial Sequence
  chemically synthesized PCR primer
tattcttatgaataataccaataccacctt

SEQ ID NO: 29 - DNA, 55 bp, Artificial Sequence
  chemically synthesized PCR primer
cccgggatggatccgtcgacaccacgtggataacgtaccattgctggaccattat

SEQ ID NO: 30 - DNA, 51 bp, Artificial Sequence
  chemically synthesized PCR primer
cccgggatggatccgccttactgacattacttgtagcattatgttttgctg

SEQ ID NO: 31 - DNA, 49 bp, Artificial Sequence
  chemically synthesized PCR primer
gtcgttacagatctgacatcagaatcataaactgactcaggaccacctg

SEQ ID NO: 32 - DNA, 51 bp, Artificial Sequence
  chemically synthesized PCR primer
gtcgttacagatctagaaccattaagagagcttttcaaagatgaggttcgt

SEQ ID NO: 33 - DNA, 56 bp, Artificial Sequence
  chemically synthesized PCR primer
gctagtcctgatatcggatccttttttgatctccgtaattaaaatctaaagagtat

SEQ ID NO: 34 - DNA, 56 bp, Artificial Sequence
  chemically synthesized PCR primer
gctagtcctagatctaagcttgtaaattctaatacttatttctggcatattttttc

SEQ ID NO: 35 - DNA, 26 bp, Artificial Sequence
  chemically synthesized PCR primer
atgacagatatacataatcataagat

SEQ ID NO: 36 - DNA, 22 bp, Artificial Sequence
  chemically synthesized PCR primer
ccacagagtatcacatccgcag

SEQ ID NO: 37 - DNA, 21 bp, Artificial Sequence
  chemically synthesized PCR primer
gggctatctggaggtgttgat

SEQ ID NO: 38 - DNA, 22 bp, Artificial Sequence
  chemically synthesized PCR primer
tgatcaccaactacacctaccg

SEQ ID NO: 39 - DNA, 22 bp, Artificial Sequence
  chemically synthesized PCR primer
ctgcggatgtgatactctgtgg

SEQ ID NO: 40 - DNA, 22 bp, Artificial Sequence
  chemically synthesized PCR primer
gataccccttagggcatctaag

SEQ ID NO: 41 - DNA, 30 bp, Artificial Sequence
  chemically synthesized PCR primer
tttatataggtaaatgttttacctgtacca

SEQ ID NO: 42 - DNA, 20 bp, Artificial Sequence
  chemically synthesized PCR primer
gccgagtttgatgctgaaaa

SEQ ID NO: 43 - DNA, 4245 bp, Artificial Sequence
  chemically synthesized pUC19 plasmid
gaattccggatgagcattcatcaggcgggcaagaatgtgaataaaggccggataaaacttgtgcttatttttctttacggtctttaaaaaggccgtaatatccagctgaacggtctggttataggtacattgagcaactgactgaaatgcctcaaaatgttctttacgatgccattgggatatatcaacggtggtatatccagtgatttttttctccattttagcttccttagctcctgaaaatctcgataactcaaaaaatacgcccggtagtgatcttatttcattatggtgaaagttggaacctcttacgtgccgatcaacgtctcattttcgccaaaagttggcccagggcttcccggtatcaacagggacaccaggatttatttattctgcgaagtgatcttccgtcacaggtatttattcggcgcaaagtgcgtcgggtgatgctgccaacttactgatttagtgtatgatggtgtttttgaggtgctccagtggcttctgtttctatcagctgtccctcctgttcagctactgacggggtggtgcgtaacggcaaaagcaccgccggacatcagcgctagcggagtgtatactggcttactatgttggcactgatgagggtgtcagtgaagtgcttcatgtggcaggagaaaaaaggctgcaccggtgcgtcagcagaatatgtgatacaggatatattccgcttcctcgctcactgactcgctacgctcggtcgttcgactgcggcgagcggaaatggcttacgaacggggcggagatttcctggaagatgccaggaagatacttaacagggaagtgagagggccgcggcaaagccgtttttccataggctccgcccccctgacaagcatcacgaaatctgacgctcaaatcagtggtggcgaaacccgacaggactataaagataccaggcgtttccccctggcggctccctcgtgcgctctcctgttcctgcctttcggtttaccggtgtcattccgctgttatggccgcgtttgtctcattccacgcctgacactcagttccgggtaggcagttcgctccaagctggactgtatgcacgaaccccccgttcagtccgaccgctgcgccttatccggtaactatcgtcttgagtccaacccggaaagacatgcaaaagcaccactggcagcagccactggtaattgatttagaggagttagtcttgaagtcatgcgccggttaaggctaaactgaaaggacaagttttggtgactgcgctcctccaagccagttacctcggttcaaagagttggtagctcagagaaccttcgaaaaaccgccctgcaaggcggttttttcgttttcagagcaagagattacgcgcagaccaaaacgatctcaagaagatcatcttattaatcagataaaatatttctagatttcagtgcaatttatctcttcaaatgtagcacctgaagtcagccccatacgatataagttgtaattctcatgtttgacagcttatcatcgataagctttaatgcggtagtttatcacagttaaattgctaacgcagtcaggcaccgtgtatgaaatctaacaatgcgctcatcgtcatcctcggcaccgtcaccctggatgctgtaggcataggcttggttatgccggtactgccgggcctcttgcgggatatcgtccattccgacagcatcgccagtcactatggcgtgctgctagcgctatatgcgttgatgcaatttctatgcgcacccgttctcggagcactgtccgaccgctttggccgccgcccagtcctgctcgcttcgctacttggagccactatcgactacgcgatcatggcgaccacacccgtcctgtggatcctctacgccggacgcatcgtggccggcatcaccggcgccacaggtgcggttgctggcgcctatatcgccgacatcaccgatggggaagatcgggctcgccacttcgggctcatgagcgcttgtttcggcgtgggtatggtggcaggccccgtggccgggggactgttgggcgccatctccttgcatgcaccattccttgcggcggcggtgctcaacggcctcaacctactactgggctgcttcctaatgcaggagtcgcataagggagagcgtcgaccgatgcccttgagagccttcaacccagtcagctccttccggtgggcgcggggcatgactatcgtcgccgcacttatgactgtcttctttatcatgcaactcgtaggacaggtgccggcagcgctctgggtcattttcggcgaggaccgctttcgctggagcgcgacgatgatcggcctgtcgcttgcggtattcggaatcttgcacgccctcgctcaagccttcgtcactggtcccgccaccaaacgtttcggcgagaagcaggccattatcgccggcatggcggccgacgcgctgggctacgtcttgctggcgttcgcgacgcgaggctggatggccttccccattatgattcttctcgcttccggcggcatcgggatgcccgcgttgcaggccatgctgtccaggcaggtagatgacgaccatcagggacagcttcaaggatcgctcgcggctcttaccagcctaacttcgatcactggaccgctgatcgtcacggcgatttatgccgcctcggcgagcacatggaacgggttggcatggattgtaggcgccgccctataccttgtctgcctccccgcgttgcgtcgcggtgcatggagccgggccacctcgacctgaatggaagccggcggcacctcgctaacggattcaccactccaagaattggagccaatcaattcttgcggagaactgtgaatgcgcaaaccaacccttggcagaacatatccatcgcgtccgccatctccagcagccgcacgcggcgcatctcgggcagcgttgggtcctggccacgggtgcgcatgatcgtgctcctgtcgttgaggacccggctaggctggcggggttgccttactggttagcagaatgaatcaccgatacgcgagcgaacgtgaagcgactgctgctgcaaaacgtctgcgacctgagcaacaacatgaatggtcttcggtttccgtgtttcgtaaagtctggaaacgcggaagtcccctacgtgctgctgaagttgcccgcaacagagagtggaaccaaccggtgataccacgatactatgactgagagtcaacgccatgagcggcctcatttcttattctgagttacaacagtccgcaccgctgtccggtagctccttccggtgggcgcggggcatgactatcgtcgccgcacttatgactgtcttctttatcatgcaactcgtaggacaggtgccggcagcgcccaacagtcccccggccacggggcctgccaccatacccacgccgaaacaagcgccctgcaccattatgttccggatctgcatcgcaggatgctgctggctaccctgtggaacacctacatctgtattaacgaagcgctaaccgtttttatcaggctctgggaggcagaataaatgatcatatcgtcaattattacctccacggggagagcctgagcaaactggcctcaggcatttgagaagcacacggtcacactgcttccggtagtcaataaaccggtaaaccagcaatagacataagcggctatttaacgaccctgccctgaaccgacgaccgggtcgaatttgctttcgaatttctgccattcatccgcttattatcacttattcaggcgtagcaccaggcgtttaagggcaccaataactgccttaaaaaaattacgccccgccctgccactcatcgcagtactgttgtaattcattaagcattctgccgacatggaagccatcacagacggcatgatgaacctgaatcgccagcggcatcagcaccttgtcgccttgcgtataatatttgcccatggtgaaaacgggggcgaagaagttgtccatattggccacgtttaaatcaaaactggtgaaactcacccagggattggctgagacgaaaaacatattctcaataaaccctttagggaaataggccaggttttcaccgtaacacgccacatcttgcgaatatatgtgtagaaactgccggaaatcgtcgtggtattcactccagagcgatgaaaacgtttcagtttgctcatggaaaacggtgtaacaagggtgaacactatcccatatcaccagctcaccgtctttcattgccatacg

(19) United States
(12) Patent Application Publication
(10) Pub. No.: US 2011/0020399 A1
(43) Pub. Date: Jan. 27, 2011

(54) Vaccines and Immunomodulatory Therapies for Tularemia

(75) Inventors: Araceli Santiago, Baltimore, MD (US); Myron M. Levine, Columbia, MD (US); Eileen M. Barry, Ellicott City, MD (US)

Correspondence Address: ROYLANCE, ABRAMS, BERDO & GOODMAN, L.L.P., 1300 19TH STREET, N.W., SUITE 600, WASHINGTON,, DC, 20036, US

(73) Assignee: UNIVERSITY OF MARYLAND, BALTIMORE, Baltimore, MD (US)

(21) Appl. No.: 12/103,979

(22) Filed: Apr. 16, 2008

Related U.S. Application Data
(60) Provisional application No. 60/923,742, filed on Apr. 16, 2007.

(51) Int. Cl. A61K 39/02; C12N 1/20; C12N 1/21; C12N 15/63; A61K 31/713; C07H 21/04; A61P 31/04

(52) U.S. Cl. 424/234.1; 435/252.1; 435/252.3; 435/320.1; 514/44 A; 536/23.1

(57) ABSTRACT

The present invention relates to attenuated strains of Francisella tularensis from which genes in the guanine nucleotide biosynthetic pathway have been deleted and to methods for making the mutants. The invention further relates to the use of the attenuated strains to make vaccines.

This application claims benefit under 35 U.S.C. §119(e) to U.S. provisional application No. 60/923,742, filed Apr. 16, 2007, the entire contents of which are herein incorporated by reference.

### STATEMENT OF GOVERNMENTAL INTEREST

This invention was made with Government support under Contract No. AI057168 awarded by the National Institutes of Health. The United States Government has certain rights in the invention.

### BACKGROUND OF THE INVENTION

1. Field of the Invention

The present invention relates to attenuated strains of Francisella tularensis from which genes in the guanine nucleotide biosynthetic pathway have been deleted and to methods for making the mutants. The invention further relates to the use of the attenuated strains as vaccines.

2. Related Art

Francisella tularensis (hereafter “Ft” or “F. tularensis” or “Francisella”) is a small non-motile, gram-negative coccobacillus that is the etiological agent of the zoonotic disease tularemia in humans. It is classified as type A or B by virulence testing in animal models as well as by microbiological characterization.

F. tularensis type A is the most highly virulent type and is found in North America, while F. tularensis type B is frequently associated with tularemia in Europe and Asia. The development of disease in humans depends on the route of bacterial entry. It has been reported that aerosol transmission has a fatality rate of 60% in untreated people with as few bacteria as 10 CFU. Since the 1950s, the incidence of tularemia in the United States has steadily declined, with only 1,368 cases of tularemia reported to CDC between 1990 and 2000 (5); however, interest in tularemia has increased in recent years due its potential use as a bioweapon (6,7). Ft has been classified as by the U.S. Centers for Disease Control and Prevention as a Category A bioterrorism agent due to its ability to spread via the airborne route, its extremely low infectious dose, and its capacity to cause severe disease and death. The high infectivity of the fully virulent strain underlies the fact that the majority of research into the pathogenesis of Ft has utilized an attenuated “Live Vaccine Strain” (LVS) (reviewed in 8,9).

Ft LVS was developed in the former Soviet Union in the 1940's by repeatedly passaging the Type B strain of Ft on agar plates and then through mice (10). While Ft LVS is attenuated in humans, it is fully virulent in mice and causes a disease that resembles human tularemia (8).

For humans, natural tularemia infection can be acquired through direct contact with infected animals or contaminated hay, consumption of contaminated food or water, inhalation of contaminated air, or by the bite of an infected insect. The infectious dose required to cause human infection varies with route of entry. While respiratory infection can result from as few as 25 inhaled organisms, one must ingest upwards of 10⁸ bacteria in order to cause glandular infection (11,12). The severity of illness is also dependent upon the route of exposure as the pneumonic form of tularemia is most severe and has the highest mortality rate, greater than 30% when untreated (4,6,13). Similar to tularemia in humans, the outcome of Ft LVS exposure in mice is dependent on both the size of the inoculum as well as the route of inoculation. When mice are injected with Ft LVS intraperitoneal, the infection is nearly always fatal as the LD₅₀ is less than 10 organisms; however, the outcome is quite different when the bacteria are introduced subcutaneously or intradermal as estimates for the LD₅₀ range from 10⁵-10⁸ bacteria (14-16). Infection by the intradermal route protects mice against a subsequent lethal intraperitoneal infection (14,15,17,18), suggesting that a protective immune response to infection is achievable. Although LVS has been demonstrated to be safe and attenuated in vaccinated volunteers, the molecular mechanism of its attenuation is unknown and it is based on a type B rather than a type A strain. Accordingly, there is a need for a new generation of F. tularensis live attenuated vaccines that have well defined genetic characteristics and can be used safely for en masse vaccination.

### SUMMARY OF THE INVENTION

The present invention is directed to strains of Francisella tularensis that cannot form de novo guanine nucleotides. In particular, the present invention is directed to strains of Francisella tularensis in which one or more elements of the biosynthetic pathway utilitized by the bacteria to produce guanine nucleotides is disrupted. In a preferred embodiment, either the guaA gene, the guaB gene, or both the guaA gene and the guaB gene are altered such that the genes are not functional and either the proteins encoded by these genes are not produced or the proteins produced have diminished or no activity.

Thus, in one embodiment the present invention is directed to a strain of F. tularensis that cannot form de novo guanine nucleotides, wherein said strain has a non-functional guaA gene. In another embodiment the present invention is directed to a strain of F. tularensis that cannot form de novo guanine nucleotides, wherein said strain has a non-functional guaB gene. In a third embodiment the present invention is directed to a strain of F. tularensis that cannot form de novo guanine nucleotides, wherein said strain has a non-functional guaA gene and has a non-functional guaB gene.

In each of the embodiments of the invention where the strain of F. tularensis has a non-functional guaA gene or guaB gene, the respective gene may be rendered non-functional due to one or more of a deletion, insertion, substitution or rearrangement of the gene.

In preferred embodiments of the invention where the strain of F. tularensis has a non-functional guaA gene or guaB gene, the strain is a recombinant strain. Also in preferred embodiments, while the strains of F. tularensis of the present invention may be derived from any strain of F. tularensis, preferably the strains of F. tularensis of the present invention are derived from F. tularensis type A, F. tularensis type B, F. tularensis Schu S4 strain or F. tularensis LVS.

The present invention is also directed to vaccines that may be used to provide protective immunity to a disease caused by F. tularensis. Such vaccines are comprised of a pharmaceutically effective amount of a strain of F. tularensis of the present invention, preferably in a formulation that further comprises a pharmaceutically acceptable carrier or diluent. In one embodiment, the present invention is directed to a vaccine formulation comprising (a) a pharmaceutically effective amount of a strain of F. tularensis that cannot form de novo guanine nucleotides, wherein said strain has a non-functional guaA gene, or a non-functional guaB gene, or both a non-functional guaA gene and has a non-functional guaB gene, and (b) a pharmaceutically acceptable carrier or diluent.

In the embodiments of the present invention directed to vaccines, preferably the pharmaceutically effective amount of a strain of F. tularensis of the present invention is about 10 to about 1×10¹¹ cfu/ml.

The present invention is also directed to methods of generating an immune response in a subject to a strain of F. tularensis of the present invention. In one embodiment, the present invention is directed to methods of generating an immune response in a subject, comprising administering an immunologically effective amount of strain of F. tularensis of the present invention to a subject, thereby generating an immune response in a subject. In another embodiment the present invention is directed to methods of generating an immune response in a subject, comprising administering an immunologically effective amount of a vaccine or vaccine formulation of the present invention to a subject, thereby generating an immune response in a subject. In each of the methods of generating an immune response of the present invention, the immune response is preferably a protective immune response.

The present invention is also directed to suicide plasmids, in particular suicide plasmids that may be used in the production of the strains of F. tularensis of the present invention. The suicide plasmids of the present invention include the pFT724 guaB suicide plasmid, the pFT695 guaA suicide plasmid, and the pFT758 guaA suicide plasmid. The suicide plasmids of the present invention include those further comprising one or more selectable markers selected from the group consisting of kanamycin (km), chloramphenicol, tetracycline, and ampicillin. Each of the suicide plasmids of the present invention may further comprise a promoter operably linked to sacB. In preferred embodiments the promoter is a native promoter of sacB or groEL, or another Francisella promoter.

The present invention is further directed to methods for treating tularemia in an animal. In one embodiment, the present invention encompasses methods for treating tularemia in an animal comprising blocking expression of the guaA gene of F. tularensis by administering to an animal in need of treatment a therapeutically effective amount of an antisense oligonucleotide that is sufficiently complementary to the guaA gene of F. tularensis to permit specific hybridization under physiologic conditions and wherein the antisense nucleic acid inhibits expression of the guaA gene, thereby treating tularemia in an animal. In a similar embodiment, the present invention encompasses methods for treating tularemia in an animal, comprising blocking expression of the guaB gene of F. tularensis by administering to an animal in need of treatment a therapeutically effective amount of an antisense oligonucleotide that is sufficiently complementary to the guaB gene of F. tularensis to permit specific hybridization under physiologic conditions and wherein the antisense nucleic acid inhibits expression of the guaB gene, thereby treating tularemia in an animal.

In a further embodiment, the present invention encompasses methods for treating tularemia in an animal, comprising blocking translation of messenger RNA produced from the guaA gene of F. tularensis by administering to an animal in need of treatment a therapeutically effective amount of an antisense oligonucleotide that is sufficiently complementary to messenger RNA produced from the guaA gene to permit specific hybridization under physiologic conditions to the messenger RNA, and wherein the antisense nucleic acid inhibits translation of the messenger RNA, thereby treating tularemia in an animal. Similarly, the present invention encompasses methods for treating tularemia in an animal, comprising blocking translation of messenger RNA produced from the guaB gene of F. tularensis by administering to an animal in need of treatment a therapeutically effective amount of an antisense oligonucleotide that is sufficiently complementary to messenger RNA produced from the guaB gene to permit specific hybridization under physiologic conditions to the messenger RNA, and wherein the antisense nucleic acid inhibits translation of the messenger RNA, thereby treating tularemia in an animal.

In yet a further embodiment, the present invention encompasses methods for preventing tularemia in an animal, comprising blocking translation of messenger RNA produced from the guaA gene of F. tularensis by administering to an animal in need of prevention a therapeutically effective amount of an antisense oligonucleotide that is sufficiently complementary to messenger RNA produced from the guaA gene to permit specific hybridization under physiologic conditions to the messenger RNA, and wherein the antisense nucleic acid inhibits translation of the messenger RNA, thereby preventing tularemia in an animal. Similarly, the present invention encompasses methods for preventing tularemia in an animal, comprising blocking translation of messenger RNA produced from the guaB gene of F. tularensis by administering to an animal in need of prevention a therapeutically effective amount of an antisense oligonucleotide that is sufficiently complementary to messenger RNA produced from the guaB gene to permit specific hybridization under physiologic conditions to the messenger RNA, and wherein the antisense nucleic acid inhibits translation of the messenger RNA, thereby preventing tularemia in an animal.

Moreover, the present invention is directed to an isolated polynucleotide molecule comprising (i) the F. tularensis guaA gene polynucleotide sequence set forth in SEQ ID NO:1, (ii) an isolated polynucleotide molecule comprising a polynucleotide sequence having at least 95% identity to the polynucleotide set forth in SEQ ID NO:1, or (iii) an isolated polynucleotide molecule comprising a polynucleotide sequence that hybridizes under stringent hybridization conditions to a complement of the polynucleotide set forth in SEQ ID NO:1.

Similarly, the present invention is directed to an isolated polynucleotide molecule comprising (i) the F. tularensis guaB gene polynucleotide sequence set forth in SEQ ID NO:2, (ii) an isolated polynucleotide molecule comprising a polynucleotide sequence having at least 95% identity to the polynucleotide set forth in SEQ ID NO:2, or (iii) an isolated polynucleotide molecule comprising a polynucleotide sequence that hybridizes under stringent hybridization conditions to a complement of the polynucleotide set forth in SEQ ID NO:2.

### BRIEF DESCRIPTION OF THE DRAWINGS

FIG. 1: is a diagram of the guaA and guaB metabolic reactions in the biosynthesis of the purine adenine monophosphate (AMP) and guanine monophosphate (GMP).

FIG. 2: depicts structural diagrams of adenine monophosphate (AMP) and guanine monophosphate (GMP).

FIG. 3: is a diagram showing the suicide plasmid pFT724 used for allelic recombination events to delete guaB in F. tularensis LVS strain.

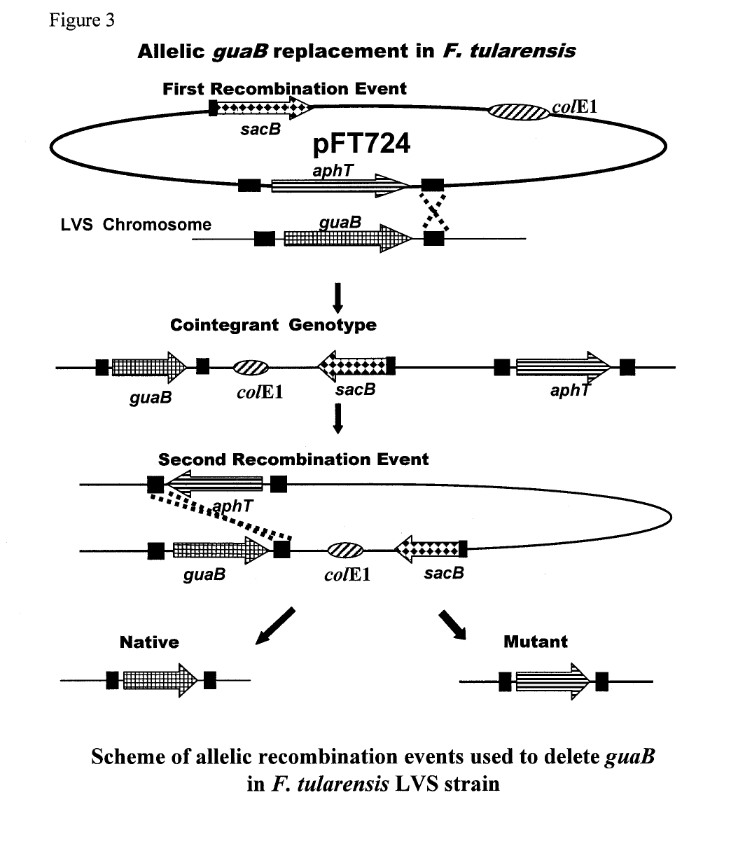

FIG. 4: is a diagram showing the suicide plasmid pFT695 used for allelic recombination events to delete guaA in F. tularensis LVS strain.

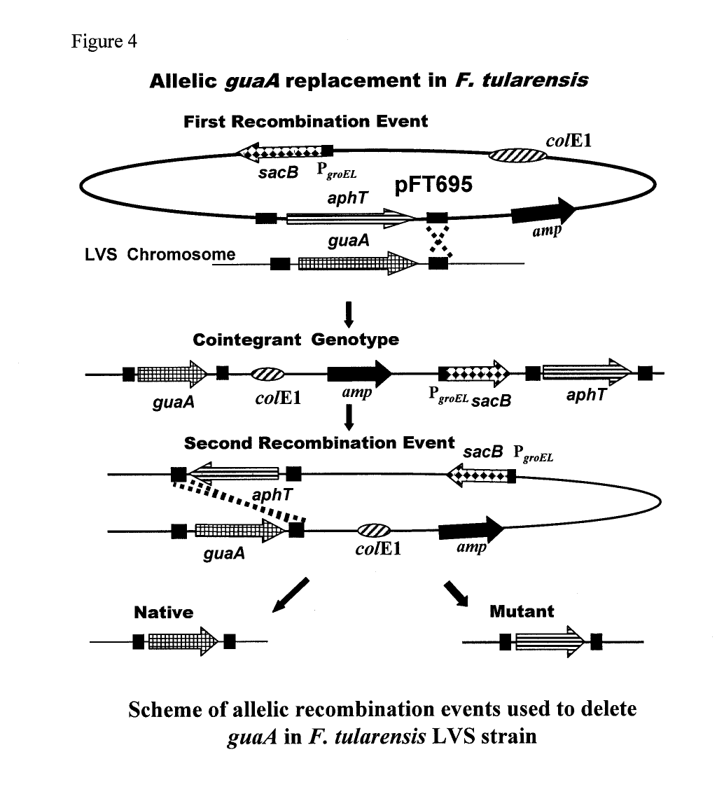

FIG. 5: Deletion of guaA (panel A) or guaB (panel B) loci in F. tularensis LVS was generated by allelic exchange between the deleted gene introduced on a suicide plasmid and the wild type chromosomal gene. F. tularensis LVS::pFT724 and LVS::pFT695 cointegrants were generated by recombination of pFT724 or pFT695 into the LVS genome. Second recombination events were identified by growing the strains in 10% sucrose which selects against the presence of the plasmid bearing the sacB gene. Colonies that were auxotrophic for guanine were isolated on MHA-B plates and evaluated by PCR. Genotypes were confirmed by PCR analysis (panel C) using genomic DNA from the following strains as templates: parental LVS (lane 1), LVS::pFT695 cointegrant (lane 2), LVS ΔguaA mutant (lane 3), LVS::pFT724 cointegrant (lane 4) and LVS ΔguaB mutant strain (lane 5). Specific primers for guaA (OLIGA1 (P528), OLIGA2 (P535), OLIGkm (P533)) panel 1C, guaB (OLIGB1 (P530), OLIGB2 (P534), OLIGkm (P533)) panel 2C, and F. tularensis strain (PRD1A, PRD1B) panel 3C (J. Clin. Microbiol. 2003, 41;2924-31) were used.

FIG. 6: demonstrates the auxotrophic phenotype of the ΔguaA and ΔguaB mutations showing their inability to grow without the addition of exogenous guanine to the media.

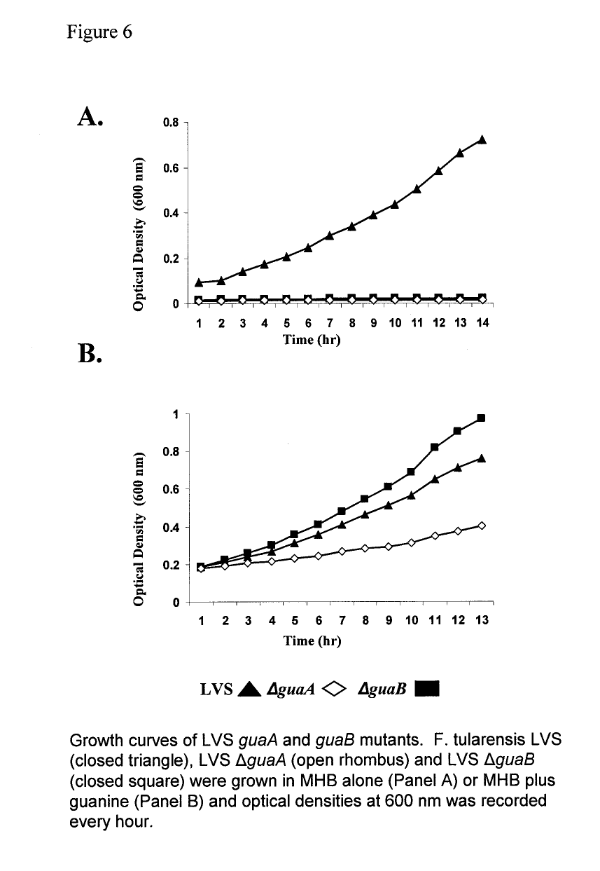

FIG. 7: demonstrates the defect in growth and survival of F. tularensis ΔguaA and ΔguaB mutants compared to the parental LVS in J774 macrophages.

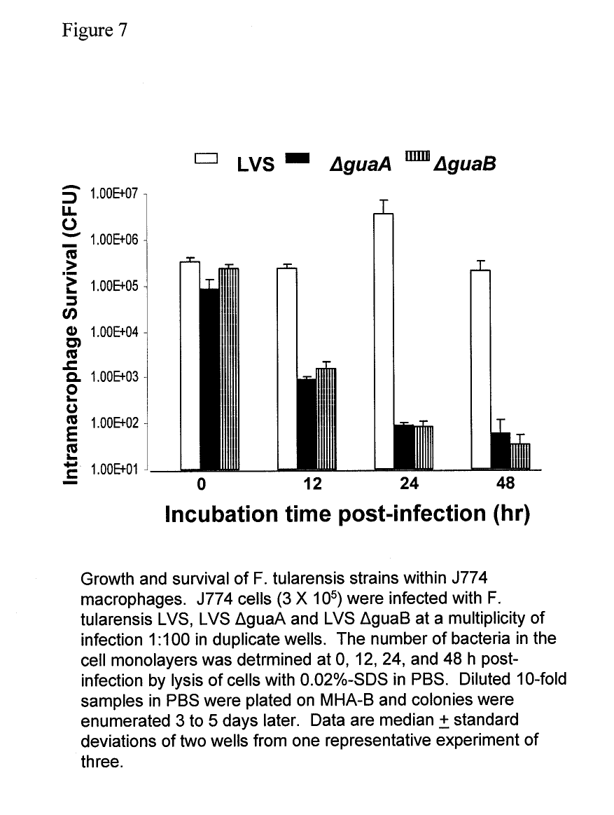

FIG. 8: demonstrates the auxotrophic phenotype of the Schu S4 ΔguaB mutations showing their inability to grow without the addition of exogenous guanine to the media

FIG. 9: demonstrates the auxotrophic phenotype of the Schu S4 ΔguaA mutations showing their inability to grow without the addition of exogenous guanine to the media

FIG. 10: demonstrates replication of Schu S4 wild type and Schu S4 ΔguaB in cultured J774 macrophages.

FIG. 11: demonstrates Schu S4 ΔguaB assessment in a mouse model of infection.

### DETAILED DESCRIPTION

Purine biosynthesis has been targeted for mutations to create live attenuated vaccines in certain enteric bacteria. For example, purA and purB mutations in Salmonella (interrupting the de novo biosynthesis of adenine nucleotides) are so highly attenuating (McFarland et al, Microb. Pathogen., 3:129-141 (1987)), that these strains are non-protective in animals (O'Callagham et al, Infect. Immun., 56:419-423 (1988)), and poorly immunogenic in humans (Levine et al, J. Clin. Invest., 79:888-902 (1987)).

In contrast, mutations in several of the genes involved in the common purine pathway (i.e., purF, purG, purC, purHD) that interrupt the biosynthesis of both purine nucleotides, or in guaB or guaA, thereby interrupting the biosynthesis of guanine nucleotides, are far less attenuating (McFarland et al, supra). It has been reported that purine negative-auxotrophs of S. flexneri 2a and S. sonnei (derived by non-specific mutagenesis) are not significantly attenuated (Linde et al, Vaccine, 8:25-29 (1990)); and guinea pigs inoculated in their conjunctival sac with these strains developed full-blown purulent keratoconjunctivitis (positive Sereny test (Sereny, Acta Microbiol. Acad. Sci. Hung., 4:367-376 (1957); and Linde et al, supra).

However, purine biosynthesis, particularly guanine nucleotide biosynthesis was successfully exploited in preparing attenuated Shigella (U.S. Pat. No. 5,783,196) and Salmonella (U.S. Pat. No. 6,190,669) vaccines, both of which are incorporated in their entirety as if set forth fully herein. The chromosomal genome of Shigella was modified by introducing a deletion into the guaBA operon, and thus blocking the de novo biosynthesis of guanine nucleotides. A non-polar mutation in the guaBA operon inactivates the purine metabolic pathway enzymes IMP dehydrogenase (encoded by guaB) and GMP synthetase (encoded by guaA). As a consequence of this mutation, Shigella mutants were unable to synthesize de novo GMP, and consequently GDP and GTP nucleotides, which severely limited growth of these auxotrophic mutants in mammalian tissues. In vitro, the guaB-A mutants of Shigella were unable to grow in minimal medium unless supplemented with guanine In tissue cultures, the guaBA Shigella mutants were found to show a significant reduction in their capability for invasion. In vivo, these mutants are dramatically attenuated, but nonetheless confer a striking protective immune response (7, 9, 13). It has been suggested that the attenuation due to the de novo synthesis of guanine nucleotides may be due to the inefficiency of the salvage pathway.

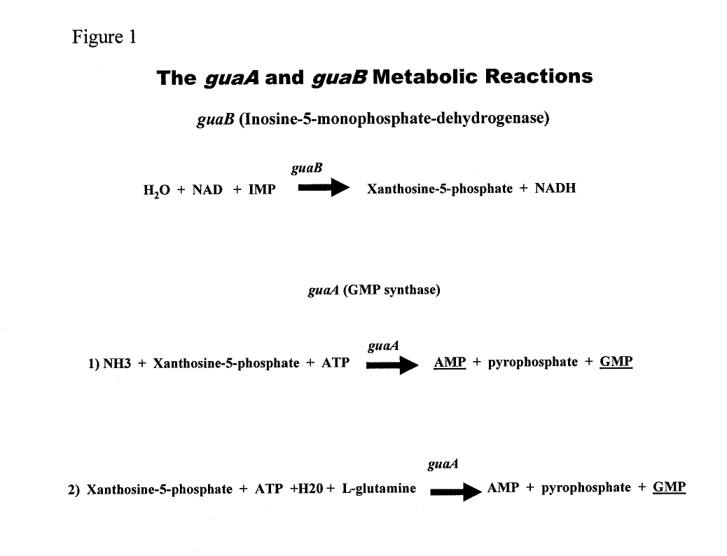

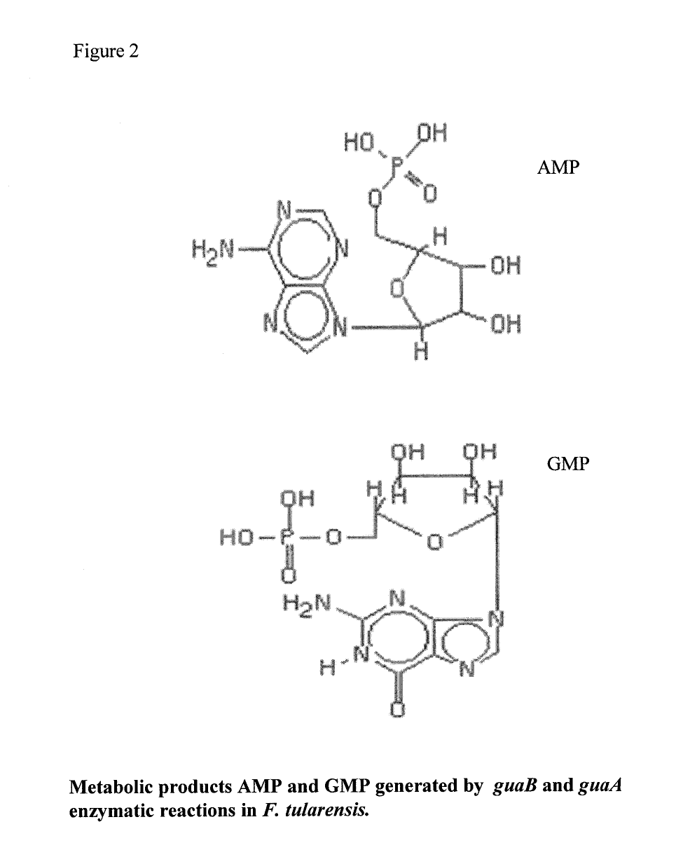

The guaB and guaA genes of F. tularensis also encode IMP dehydrogenase and GMP synthetase respectively, two critical enzymes in the purine biosynthetic pathway. Part of this pathway involving guaA and guaB in adenine monophosphate (AMP) and guanine monophosphate (GMP) biosynthesis is diagrammed in FIG. 1. The molecular structures of adenine monophosphate (AMP) and guanine monophosphate (GMP) are shown in FIG. 2.

In Shigella, the guaB and guaA genes are contained within a single operon structure and it was possible to make a single mutation that deleted both genes. By contrast, the guaB and guaA genes of F. tularensis are encoded separately and at different loci within the chromosome. Further complicating matters is the observation that not all mutations affecting guanine nucleotide synthesis are attenuating, for example, guanine nucleotide synthesis mutants in Salmonella are only minimally attenuating (McFarland et al, supra).

The amino acid sequences encoded by the guaB and guaA genes are only 54% and 61% identical respectively between the enteric (E. coli) and the nonenteric bacterium Francisella, while they are 99% identical between enteric organisms including Shigella and Salmonella. Another distinction is that the guaB and guaA genes are part of a single operon in enteric organisms, while in F. tularensis they are distinct loci widely separated on the chromosome (312,613 bp). Genetic manipulation of F. tularensis has only just begun to be worked out. The genes for guaA and guaB are set forth in SEQ ID NO:1 and SEQ ID NO:2, respectively.

The work is further complicated by the fact that plasmids that replicate in enteric organisms do not replicate in F. tularensis. Therefore, specialized “suicide” plasmids described below for use in allelic exchange and genetic manipulation had to be generated to make guaA and guaB mutants for F. tularensis. Moreover, F. tularensis is particularly difficult to work with because it is a fastidious organism that requires special growth media. Difficulty growing F. tularensis may be related to its lifecycle since it grows in macrophages in the mammalian host and is believed to grow in amoeba in the environment.

The presence of two independently attenuating mutations in genes that are widely separated on the chromosome (312,613 base pairs) offers safety against the chance of reversion to the wild type virulent strain therefore a double ΔguaA or ΔguaB deletion mutant is a preferred embodiment.

Strains of Francisella tularensis

The present invention is thus directed to strains of Francisella tularensis that cannot form de novo guanine nucleotides. In particular, the present invention is directed to strains of Francisella tularensis in which one or more elements of the biosynthetic pathway utilitized by the bacteria to produce guanine nucleotides is disrupted. In a preferred embodiment, either the guaA gene, the guaB gene, or both the guaA gene and the guaB gene are altered such that the genes are not functional and either the proteins encoded by these genes are not produced or the proteins produced have diminished or no activity. Such strains are also referred to herein as attenuated ΔguaA and/or ΔguaB strains of F. tularensis.

Thus, in one embodiment the present invention is directed to a strain of F. tularensis that cannot form de novo guanine nucleotides, wherein said strain has a non-functional guaA gene. In another embodiment the present invention is directed to a strain of F. tularensis that cannot form de novo guanine nucleotides, wherein said strain has a non-functional guaB gene. In a third embodiment the present invention is directed to a strain of F. tularensis that cannot form de novo guanine nucleotides, wherein said strain has a non-functional guaA gene and has a non-functional guaB gene.

In each of the embodiments of the invention where the strain of F. tularensis has a non-functional guaA gene or guaB gene, the particular alteration that results in a non-functional guaA gene or guaB gene is not important. Therefore, the particular gene may be rendered non-functional due to an alteration to the gene such as one or more of a deletion, insertion, substitution or rearrangement of the gene. The alteration may be in the coding region of the gene, in a non-coding region of the gene, or in a region controlling the transcription or translation of the gene. The alteration may also be in a region of the bacterial genome not associated with the gene, but where the transcription or translation of the coding region encompassed by the gene is changed. The alteration may result in diminished production of the polypeptide encoded by the gene or a complete abolishment of production of the polypeptide encoded by the gene. Alternatively, the alteration may allow normal, reduced or increased levels of the polypeptide encoded by the gene, but result in a polypeptide with diminished activity, increased activity or no activity.

In a preferred embodiment, the strains of F. tularensis of the present invention have a deletion in the guaA gene, the guaB gene, or both the guaA gene and the guaB gene. In a further preferred embodiment, the strains of F. tularensis of the present invention have a deletion in the guaA gene, the guaB gene, or both the guaA gene and the guaB gene, and the production of the polypeptides encoded by the genes (GMP synthetase and IMP dehydrogenase, respectively) is reduced or abolished. In another preferred embodiment, the strains of F. tularensis of the present invention have a deletion in the guaA gene, the guaB gene, or both the guaA gene and the guaB gene, and a non-functional polypeptide encoded by the genes is produced.

Inactivation of the guaA gene and/or guaB gene can also be carried out by an insertion of foreign DNA, or by site-directed mutagenesis with oligonucleotides (Sambrook et al, supra) so as to interrupt the correct transcription or translation of mRNA encoded by guaB and/or guaA. The typical size of an insertion that can inactivate the guaA gene and guaB gene is from 1 base pair to 100 kbp. The insertion can be made anywhere inside the guaA gene, guaB gene coding region or between the coding region and the promoter or within the promoter or other control elements.

The strains of F. tularensis of the present invention also include those strains that produce diminished levels of the polypeptide encoded by the guaA and/or guaB genes or produce no polypeptide, due to an alteration of a sequence in the strain other than the sequence of the guaA or guaB gene. For example, such strains include those strains having an alteration in the coding region of a protein required for transcription or translation of the sequence encoding the polypeptide encoded by the guaA and/or guaB genes or maturation of the polypeptide.

The strains of F. tularensis of the present invention further include those strains that produce diminished levels of GMP synthetase and/or IMP dehydrogenase, or produce no GMP synthetase and/or IMP dehydrogenase due to the introduction into the strain of a polynucleotide, the expression of which results in the production of a protein that alters the production levels of the polypeptides.

In one embodiment, the strains of F. tularensis of the present invention include those strains where the amount of GMP synthetase and/or IMP dehydrogenase produced is decreased relative to the amount of polypeptide produced by the strain of F. tularensis from which the mutated strain was produced. Such a decrease may be a decrease of 1%, 5%, 10%, 15%, 20%, 25%, 30% 40%, 50%, 60%, 70%, 80%, 90%, 95%, 96%, 97%, 98%, 99% or 100%. In another embodiment, the strains of F. tularensis of the present invention include those where the activity of GMP synthetase and/or IMP dehydrogenase is decreased relative to the activity of the polypeptide in the strain of F. tularensis from which the mutated strain was produced. Such a decrease may be a decrease of 1%, 5%, 10%, 15%, 20%, 25%, 30% 40%, 50%, 60%, 70%, 80%, 90%, 95%, 96%, 97%, 98%, 99%, or 100%.

The particular means used to create the mutated strains described herein will be readily apparent to one of skill in the art. Particular means for creating the mutated strains is provided in the Examples below.

In preferred embodiments of the invention where the strain of F. tularensis has a non-functional guaA gene or guaB gene, the strain is a recombinant strain. Also in preferred embodiments, while the strains of F. tularensis of the present invention may be derived from any strain of F. tularensis, preferably the strains of F. tularensis of the present invention are derived from F. tularensis type A, F. tularensis type B, F. tularensis Schu S4 strain or F. tularensis LVS.

Preferably each of the strains of F. tularensis of the present invention is auxotrophic.

Vaccines

The present invention is also directed to vaccines that may be used to provide protective immunity to a disease caused by F. tularensis. Such vaccines are comprised of a pharmaceutically effective amount of a strain of F. tularensis of the present invention, preferably in a formulation that further comprises a pharmaceutically acceptable carrier or diluent. In one embodiment, the present invention is directed to a vaccine formulation comprising (a) a pharmaceutically effective amount of a strain of F. tularensis that cannot form de novo guanine nucleotides, wherein said strain has a non-functional guaA gene, or a non-functional guaB gene, or both a non-functional guaA gene and has a non-functional guaB gene, and (b) a pharmaceutically acceptable carrier or diluent.

Preferably, in each of the vaccines and vaccine formulations of the present invention the strain of F. tularensis in the vaccine or vaccine formulation is living, although the vaccines and vaccine formulations of the present invention also encompass vaccines and vaccine formulations comprising strains of F. tularensis that are further attenuated or dead, or a mixture thereof.

The vaccines and vaccine formulations described herein can be formulated in a variety of useful formats for administration by a variety of routes. Concentrations of the strains of F. tularensis in the vaccines and formulations described herein will be such that a pharmaceutically effective amount of a strain of F. tularensis is included in the vaccine or formulation. Determination of such a concentration would be readily apparent to those of ordinary skill in the art. However, certain embodiments of the present invention the pharmaceutically effective amount of a strain of F. tularensis of the present invention is preferably about 10 to about 1×10¹¹ cfu/ml. In preferred embodiments the pharmaceutically effective amount of a strain of F. tularensis is about 1×10² cfu/ml, about 1×10³ cfu/ml, about 1×10⁴ cfu/ml, about 1×10⁵ cfu/ml, about 1×10⁶ cfu/ml, about 1×10⁷ cfu/ml, about 1×10⁸ cfu/ml, about 1×10⁹ cfu/ml, about 1×10¹⁰ cfu/ml, about 1×10¹¹ cfu/ml, or about 1×10¹² cfu/ml.

Administration of the vaccines and vaccine formulations can be by any form generally used in the art, and includes nasal application, by inhalation, ophthalmically, orally, rectally, vaginally, or by any other mode that results in the vaccines contacting mucosal tissues.

Solid formulations of the vaccines for oral administration may contain suitable carriers or excipients, such as corn starch, gelatin, lactose, acacia, sucrose, microcrystalline cellulose, kaolin, mannitol, dicalcium phosphate, calcium carbonate, sodium chloride, or alginic acid. Disintegrators that can be used include, without limitation, micro-crystalline cellulose, cornstarch, sodium starch glycolate, and alginic acid. Tablet binders that may be used include acacia, methylcellulose, sodium carboxymethylcellulose, polyvinylpyrrolidone, hydroxypropyl methylcellulose, sucrose, starch, and ethylcellulose. Lubricants that may be used include magnesium stearates, stearic acid, ailicone fluid, talc, waxes, oil, and colloidal silica.

In one embodiment of the present invention, the vaccines and vaccine formulations exist as atomized dispersions for delivery by inhalation. The atomized dispersion of the vaccine formulation typically contains carriers common for atomized or aerosolized dispersions, such as buffered saline and/or other compounds well known to those of skill in the art. The delivery of the vaccines and vaccine formulations via inhalation has the effect of rapidly dispersing the vaccine and vaccine formulation to a large area of mucosal tissues as well as quick absorption by the blood for circulation of the strains of F. tularensis of the present invention. One example of a method of preparing an atomized dispersion is described in U.S. Pat. No. 6,187,344, entitled, “Powdered Pharmaceutical Formulations Having Improved Dispersibility,” which is hereby incorporated by reference in its entirety.

The vaccines and vaccine formulations described herein can also be formulated in the form of a rectal or vaginal suppository. Typical carriers used in the formulation of the inactive portion of the suppository include polyethylene glycol, glycerine, cocoa butter, and/or other compounds well known to those of skill in the art. Although not wishing to be bound by theory, delivery of vaccines and vaccine formulations via a suppository is hypothesized to have the effect of contacting a mucosal surface with the strains of F. tularensis of the present invention for release to proximal mucosal tissues. Distal mucosal tissues also receive the strains of F. tularensis of the present invention by diffusion. Other suppository formulations suitable for delivery of the vaccines and vaccine formulations encompassed by the present invention are also contemplated.

Additionally, the vaccines and vaccine formulations may also be administered in a liquid form. The liquid can be for oral dosage, for ophthalmic or nasal dosage as drops, or for use as an enema or douche. When the vaccine formulation is formulated as a liquid, the liquid can be either a solution or a suspension of the vaccine formulation. There are a variety of suitable formulations for the solution or suspension of the vaccine formulation that are well know to those of skill in the art, depending on the intended use thereof. Liquid formulations for oral administration prepared in water or other aqueous vehicles may contain various suspending agents such as methylcellulose, alginates, tragacanth, pectin, kelgin, carrageenan, acacia, polyvinylpyrrolidone, and polyvinyl alcohol. The liquid formulations may also include solutions, emulsions, syrups and elixirs containing, together with the active compound(s), wetting agents, sweeteners, and coloring and flavoring agents. Various liquid and powder formulations can be prepared by conventional methods for inhalation into the lungs of the mammal to be treated.

Delivery of the described vaccines and vaccine formulations in liquid form via oral dosage exposes the mucosa of the gastrointestinal and urogenital tracts to the strains of F. tularensis of the present invention. A suitable dose, stabilized to resist the pH extremes of the stomach, delivers the vaccine or vaccine formulation to all parts of the gastrointestinal tract, especially the upper portions thereof. Any method of stabilizing the vaccine in a liquid oral dosage such that the effective delivery of the vaccine formulation is distributed along the gastrointestinal tract is contemplated for use with the vaccine formulations described herein.

Delivery of the described vaccines and vaccine formulations in liquid form via ophthalmic drops exposes the mucosa of the eyes and associated tissues to the strains of F. tularensis of the present invention. A typical liquid carrier for eye drops is buffered and contains other compounds well known and easily identifiable to those of skill in the art.

Delivery of the described vaccines and vaccine formulations in liquid form via nasal drops exposes the mucosa of the nose and sinuses and associated tissues to the strains of F. tularensis of the present invention. Liquid carriers for nasal drops are typically various forms of buffered saline.

Administration of the vaccines and vaccine formulations may also be via non-mucosal routes such as intravenous, intraperitoneal, intramuscular, subcutaneous and intradermal routes, among others.

Injectable formulations of the vaccine formulation may contain various carriers such as vegetable oils, dimethylacetamide, dimethylformaamide, ethyl lactate, ethyl carbonate, isopropyl myristate, ethanol, polyols (glycerol, propylene glycol, and liquid polyethylene glycol) and the like. Intramuscular preparations can be prepared and administered in a pharmaceutical excipient such as Water-for-Injection, 0.9% saline, or 5% glucose solution.

Methods of Generating an Immune Response

The present invention is also directed to methods of generating an immune response in a subject to a strain of F. tularensis of the present invention. In one embodiment, the present invention is directed to methods of generating an immune response in a subject, comprising administering an immunologically effective amount of a strain of F. tularensis of the present invention to a subject, thereby generating an immune response in a subject. In another embodiment the present invention is directed to methods of generating an immune response in a subject, comprising administering an immunologically effective amount of a vaccine or vaccine formulation of the present invention to a subject, thereby generating an immune response in a subject. In each of the methods of generating an immune response of the present invention, the immune response is preferably a protective immune response.

The strains of F. tularensis of the present invention, and the vaccines and vaccine formulations, are administered in a pharmaceutically acceptable form and in substantially non-toxic quantities. In particular, strains of F. tularensis of the present invention and the vaccines and vaccine formulations can be administered in amounts appropriate to those individual strains included in the composition to produce an immune response (an immunologically effective amount). Appropriate doses can readily be determined by techniques well known to those of ordinary skill in the art without undue experimentation. Such a determination will be based, in part, on the tolerability and efficacy of a particular dose.

In certain embodiments, the highest dose allowable of the strains of F. tularensis and vaccines and vaccine formulations of the present invention for use in the methods of generating an immune response may be determined by LD₅₀ studies. This allows determination of the optimal route and amount of administration resulting in the strongest immune responses. However, certain embodiments of the present invention are directed to strains of F. tularensis and vaccines and vaccine formulations wherein the immunologically effective amount of a strain of F. tularensis of the present invention is preferably about 10 to about 1×10¹¹ cfu/ml. In preferred embodiments the immunologically effective amount of a strain of F. tularensis is about 1×10² cfu/ml, about 1×10³ cfu/ml, about 1×10⁴ cfu/ml, about 1×10⁵ cfu/ml, about 1×10⁶ cfu/ml, about 1×10⁷ cfu/ml, about 1×10⁸ cfu/ml, about 1×10⁹ cfu/ml, about 1×10¹⁰ cfu/ml, about 1×10¹¹ cfu/ml, or about 1×10¹² cfu/ml.

The strains of F. tularensis and vaccines and vaccine formulations may be administered in a single dose or in multiple doses over prolonged periods of time. In particular, the strains of F. tularensis and the vaccines and vaccine formulations may be administered for periods up to about one week, and even for extended periods longer than one month or one year. In some instances, administration of the compounds may be discontinued and resumed at a later time. For example, a second dose can be administered 28 days later, or at some other time interval to be determined. Serum for antibody assessment can be collected prior to immunization and fourteen days following each dose. Sera is assessed for antibodies against killed bacteria and an aqueous ether bacterial extract demonstrated to be superior antigen for response evaluation (12).

The attenuated ΔguaB and/or ΔguaA strains of F. tularensis of the present invention can be tested for immunogenicity and protective capacity in the mouse model using methods well known to those skilled in the art. The mouse model has been used extensively to study immunity to Francisella (5, 6).

A kit comprising the necessary components of a vaccine or vaccine formulation that elicits an immune response to a selected strain of F. tularensis and instructions for its use is also within the purview of the present invention.

Suicide Plasmids

The present invention is also directed to suicide plasmids, in particular suicide plasmids that may be used in the production of the strains of F. tularensis of the present invention.

Suicide plasmids are plasmids that do not replicate in the target bacteria. They are used to introduce mutated versions of homologous sequences into the target organism. Introduction of a suicide plasmid into a target bacteria followed by selection for the antibiotic resistance encoded by the plasmid selects for those bacteria into which the plasmid has integrated by homologous recombination within the homologous sequences. In general, two regions of homologous DNA are included which flank the region on the target organism to be deleted. Selection for a second recombination event wherein the plasmid excises from the target organism's genome allows exchange of the deleted version of the gene for the wild type version in the bacteria. Selection for excision of the plasmid from the bacterial genome is accomplished herein with the use of the sacB gene and growth on sucrose.

Novel suicide plasmids of the present invention were designed for use in Francisella strains (outlined in detail in the Examples). For example, a suicide plasmid for making the ΔguaB strain is pFT724. Other plasmids were developed to generate a deletion in guaA. Plasmid pFT695 was used to create the ΔguaA strain described herein. Another plasmid, pFT758, was created with the kanamycin resistance cassette outside of the deleted region to allow a final unmarked chromosome guaA deletion and the removal of the ampicillin resistance marker (a requirement for use in a Type A strain). Other plasmids created include, for example, guaB and guaA suicide plasmids for Schu S4 (pFT840 and pFT894). Using the suicide plasmids and allelic exchange technology strains of LVS and Schu S4 bacteria having guaA or guaB deletions (as also used herein: ΔguaA and ΔguaB) were made as is described in more detail in the examples.

The suicide plasmids of the present invention include the pFT724 guaB suicide plasmid, the pFT695 guaA suicide plasmid, the pFT840 guaB suicide plasmid, the pFT894 guaA suicide plasmid and the pFT758 guaA suicide plasmid. The suicide plasmids of the present invention include those further comprising one or more selectable markers selected from the group consisting of kanamycin (km), chloramphenicol, tetracycline, and ampicillin. Each of the suicide plasmids of the present invention may further comprise a promoter operably linked to sacB. In preferred embodiments the promoter is a native promoter of sacB or groEL, or another Francisella promoter.

Antisense Oligonucleotides

Other embodiments of the present invention include antisense oligonucleotides that substantially hybridize to the guaA or guaB gene and thereby interfere with their expression. These antisense oligonucleotides can be used therapeutically to treat or prevent tularemia by blocking expression of the guaA or guaB gene, or by inhibiting translation of the mRNA that encodes the guaA or guaB gene products. In another embodiment, small interfering RNA is used to block expression of the guaA or guaB gene products thereby treating or preventing tularemia. Antisense-RNA and antisense-DNA have been used therapeutically in mammals to treat various diseases. See for example Agrawal, S. and Zhao, Q. (1998) Curr. Opin. Chemical Biol. Vol. 2, 519-528; Agrawal, S and Zhang, R. (1997) CIBA Found. Symp. Vol. 209, 60-78; and Zhao, Q, et al., (1998), Antisense Nucleic Acid Drug Dev. Vol 8, 451-458; the entire contents of which are hereby incorporated by reference as if fully set forth herein.

Antisense oligodeoxyribonucleotides (antisense-DNA) and oligoribonucleotides (antisense-RNA) can base pair with a gene, or its mRNA transcript. An antisense PS-oligodeoxyribonucleotide for treatment of cytomegalovirus retinitis in AIDS patients is the first antisense oligodeoxyribonucleotiede approved for human use in the US. Anderson, K. O., et al., (1996) Antimicrobiol. Agents Chemother. Vol. 40, 2004-2011, the entire contents of which are hereby incorporated by reference as if fully set forth herein.

U.S. Pat. No. 6,828,151 by Borchers et al., entitled Antisense modulation of hematopoietic cell protein tyrosine kinase expression, describes methods for making and using antisense-oligonucleotides and their formulation. The entire contents of U.S. Pat. No. 6,828,151 is hereby incorporated by reference as if fully set forth herein. Others have shown that antisense oligonucleotides complementary to the gene for glutamine synthetase mRNA in Mtb effectively enter the bacteria, complex with the mRNA and inhibit glutamine synthetase expression, the amount of the poly-L-glutamate/glutamine component in the cell wall, and bacterial replication in vitro. Harth, G., et al., PNAS Jan. 4, 2000, Vol. 97, No. 1, P 418-423, the entire contents of which are hereby incorporated by reference as if fully set forth herein.

In yet other embodiments the guaA and/or guaB genes are used in high throughput screening assays to identify compounds that bind to and inhibit expression of the genes. Such compounds have therapeutic use to treat or prevent tularemia. In an embodiment of the present invention, detection of such compounds is facilitated by coupling the gene, or a homologue thereof, to a suitable reporter, e.g. a fluorescent reporter molecule. Suitable screening systems include, but are not limited to Northern blots, RT-PCR using specific primers and probes for the gene, solution hybridization and RNAase protection assays. Large scale screening, e.g., so called high throughput screening (HTS) of chemical and/or biologic libraries can be performed with a reporter system as described above. In another embodiment the assay for compounds that regulate gene expression is constructed as an assay suitable for high throughput (HTP) screening, for example an assay adapted for the commonly used 96-well format, the 384-well format or denser formats, such as micro arrays or chips, carrying immobilized reagents on their surface. Messenger RNA for the 44 kd isoform can also be used in a screening assay.

Antisense-RNA and antisense-DNA have been used therapeutically in mammals to treat various diseases. See for example Agrawal, S. and Zhao, Q. (1998) Curr. Opin. Chemical Biol. Vol. 2, 519-528; Agrawal, S and Zhang, R. (1997) CIBA Found. Symp. Vol. 209, 60-78; and Zhao, Q, et al., (1998), Antisense Nucleic Acid Drug Dev. Vol 8, 451-458; the entire contents of which are hereby incorporated by reference as if fully set forth herein.

The present invention is directed in part to inhibiting expression or activity of the guaA and/or guaB gene by inhibiting transcription, for example, using antisense technology. However, as would be appreciated by the skilled practitioner, any other suitable method may be utilized. As used herein, the term “antisense nucleotide” or “antisense” describes a nucleic acid including an oligoribonucleotide, oligodeoxyribonucleotide, modified oligoribonucleotide, or modified oligodeoxyribonucleotide which specifically hybridizes under physiological conditions to a gene of the invention or to an mRNA transcript of the gene and, thereby, inhibits the transcription of that gene and/or translation of mRNA. Antisense technology can be used to control gene expression through triple-helix formation of antisense DNA or RNA, both of which methods are based on binding of a polynucleotide to DNA or RNA. For example, the 5′ coding portion or the mature protein sequence, which encodes for the protein of the present invention, is used to design an antisense RNA nucleic acid of from 10 to 500 base pairs in length, preferably from 10-100, most preferably from 10-50. The only limit on the size of the antisense is its ability to hybridize with the gene or mRNA and inhibit expression of the guaA or guaB gene or ABC transporters. The antisense RNA nucleic acid specifically hybridizes to the mRNA in vivo and inhibits translation of an mRNA molecule into the protein (antisense—Okano, J. Neurochem., 56:560 (1991); Oligodeoxynucleotides as Antisense Inhibitors of Gene Expression, CRC Press, Boca Raton, Fla. (1988)). A DNA nucleic acid is designed to be complementary to a region of the gene involved in transcription (triple-helix—see Lee et al. Nucl. Acids Res., 6:3073 (1979); Cooney et al., Science, 241:456 (1988); and Dervan et al., Science, 251: 1360 (1991), thereby preventing transcription and the production of the polypeptide.

Methods of making antisense-nucleic acids are well known in the art. Further provided are methods of modulating the expression of the guaA and guaB gene and mRNA in cells or tissues by contacting the cells or tissues with one or more of the antisense compounds or compositions of the invention. As used herein, the terms “target nucleic acid” encompass DNA encoding the guaA and guaB gene, RNA (including pre-mRNA and mRNA) transcribed from such DNA, and also cDNA derived from such RNA. The specific hybridization of an oligomeric compound with its target nucleic acid interferes with the normal function of the target nucleic acid. This modulation of function of a target nucleic acid by compounds which specifically hybridize to it is generally referred to as “antisense”. The functions of DNA to be interfered with include replication and transcription. The functions of RNA to be interfered with include all vital functions such as, for example, translocation of the RNA to the site of protein translation, translation of protein from the RNA, and catalytic activity which may be engaged in or facilitated by the RNA. The overall effect of such interference with target nucleic acid function is modulation of the expression of the guaA and guaB gene. In the context of the present invention, “modulation” means (inhibition) in the expression of the guaA and/or guaB gene. In the context of the present invention, inhibition is the preferred form of modulation of gene expression and the gene and mRNA are preferred targets.

The antisense nucleic acids of the present invention are specifically targeted to the guaA and guaB genes and the mRNA for it. The sequence of the sense or coding strand of the guaA and guaB genes is shown in SEQ ID NOs:1 and 2, respectively. The targeting process includes determination of a site or sites within the target gene for the antisense interaction to occur such that the desired inhibitory effect. Within the context of the present invention, a preferred intragenic site is the region encompassing the translation initiation or termination codon of the open reading frame (ORF) of the gene. The translation initiation codon is typically 5′-AUG (in transcribed mRNA molecules; 5′-ATG in the corresponding DNA molecule). It is also known in the art that eukaryotic and prokaryotic genes may have two or more alternative start codons, any one of which may be preferentially utilized for translation initiation in a particular cell type or tissue, or under a particular set of conditions. In the context of the invention, “start codon” and “translation initiation codon” refer to the codon or codons that are used in vivo to initiate translation of an mRNA molecule transcribed from a gene.

It is also known in the art that a translation termination codon (or “stop codon”) of a gene may have one of three sequences, i.e., 5′-UAA, 5′-UAG and 5′-UGA (the corresponding DNA sequences are 5′-TAA, 5′-TAG and 5′-TGA, respectively). The terms “start codon region” and “translation initiation codon region” refer to a portion of such an mRNA or gene that encompasses from about 25 to about 50 contiguous nucleotides in either direction (i.e., 5′ or 3′) from a translation initiation codon. Similarly, the terms “stop codon region” and “translation termination codon region” refer to a portion of such an mRNA or gene that encompasses from about 25 to about 50 contiguous nucleotides in either direction (i.e., 5′ or 3′) from a translation termination codon.

The open reading frame (ORF) or “coding region,” which is known in the art to refer to the region between the translation initiation codon and the translation termination codon, is also a region which may be targeted effectively with antisense nucleic acids or short-interfering RNA. Other target regions include the 5′ untranslated region (5′UTR), known in the art to refer to the portion of an mRNA in the 5′ direction from the translation initiation codon, and thus including nucleotides between the 5′ cap site and the translation initiation codon of an mRNA or corresponding nucleotides on the gene, and the 3′ untranslated region (3′UTR), known in the art to refer to the portion of an mRNA in the 3′ direction from the translation termination codon, and thus including nucleotides between the translation termination codon and 3′ end of an mRNA or corresponding nucleotides on the gene.

It is also known in the art that variants can be produced through the use of alternative signals to start or stop transcription and that pre-mRNAs and mRNAs can possess more that one start codon or stop codon. Variants that originate from a pre-mRNA or mRNA that use alternative start codons are known as “alternative start variants” of that pre-mRNA or mRNA. Those transcripts that use an alternative stop codon are known as “alternative stop variants” of that pre-mRNA or mRNA. One specific type of alternative stop variant is the “polyA variant” in which the multiple transcripts produced result from the alternative selection of one of the “polyA stop signals” by the transcription machinery, thereby producing transcripts that terminate at unique polyA sites.

Once one or more target sites have been identified, nucleic acids are chosen that are sufficiently complementary to the target, i.e., specifically hybridizes to give the desired effect of inhibiting gene expression and transcription under physiologic conditions where the nucleic acids are used therapeutically or prophylactically.

In the context of this invention, “hybridization” means hydrogen bonding, which may be Watson-Crick, Hoogsteen or reversed Hoogsteen hydrogen bonding, between complementary nucleoside or nucleotide bases. For example, adenine and thymine are complementary nucleotides which pair through the formation of hydrogen bonds. “Complementary,” as used herein, refers to the capacity for precise pairing between two nucleotides. For example, if a nucleotide at a certain position of an nucleic acid is capable of hydrogen bonding with a nucleotide at the same position of a DNA or RNA molecule, then the nucleic acid and the DNA or RNA are considered to be complementary to each other at that position. The nucleic acid and the DNA or RNA are complementary to each other when a sufficient number of corresponding positions in each molecule are occupied by nucleotides which can hydrogen bond with each other. Thus, “specifically hybridizable” and “complementary” are terms which are used to indicate a sufficient degree of complementarity or precise pairing such that stable and specific binding occurs between the nucleic acid and the DNA or RNA target.

It is understood in the art that the sequence of an antisense compound need not and is often not 100% complementary to that of its target nucleic acid to be specifically hybridizable. An antisense compound is specifically hybridizable when binding of the compound to the target DNA or RNA molecule interferes with the normal function of the target DNA or RNA to cause a loss of utility, and there is a sufficient degree of complementarity to avoid non-specific binding of the antisense compound to non-target sequences under conditions in which specific binding is desired, i.e., under physiological conditions in the case of in vivo assays or therapeutic treatment, and in the case of in vitro assays, under conditions in which the assays are performed.

Antisense and other compounds of the invention that specifically hybridize to the target and inhibit expression of the target are identified through routine experimentation, and the sequences of these compounds are herein below identified as preferred embodiments of the invention. The target sites to which these preferred sequences are complementary are herein below referred to as “active sites” and are therefore preferred sites for targeting. Antisense compounds are commonly used as research reagents and diagnostics. For example, antisense nucleic acids, which are able to inhibit gene expression with exquisite specificity, are often used by those of ordinary skill to elucidate the function of particular genes. Antisense compounds are also used, for example, to distinguish between functions of various members of a biological pathway. Antisense modulation has, therefore, been harnessed for research use.

The specificity and sensitivity of antisense is also harnessed by those of skill in the art for therapeutic uses. Antisense nucleic acids have been employed as therapeutic moieties in the treatment of disease states in animals and man. Antisense nucleic acid drugs, including ribozymes, have been safely and effectively administered to humans and numerous clinical trials are presently underway. It is thus established that nucleic acids can be useful therapeutic modalities that can be configured to be useful in treatment regimes for treatment of cells, tissues and animals, especially humans.

While antisense nucleic acids are a preferred form of antisense compound, the present invention comprehends other oligomeric antisense compounds, including but not limited to nucleic acid mimetics. The antisense compounds in accordance with this invention preferably comprise from about 8 to about 50 nucleobases (i.e. from about 8 to about 50 linked nucleosides). Particularly preferred antisense compounds are antisense nucleic acids, even more preferably those comprising from about 12 to about 30 nucleobases. Antisense compounds include ribozymes, external guide sequence (EGS) nucleic acids (oligozymes), and other short catalytic RNAs or catalytic nucleic acids which specifically hybridize to the target nucleic acid and modulate its expression.

The antisense compounds used in accordance with this invention may be conveniently and routinely made through the well-known technique of solid phase synthesis. Equipment for such synthesis is sold by several vendors including, for example, Applied Biosystems. (Foster City, Calif.). Any other means for such synthesis known in the art may additionally or alternatively be employed. It is well known to use similar techniques to prepare nucleic acids such as the phosphorothioates and alkylated derivatives.

The antisense compounds of the invention are synthesized in vitro and do not include antisense compositions of biological origin. Certain embodiments do cover genetic vector constructs designed to direct the in vivo synthesis of antisense molecules in specifically targeted cells like cancer cells. The compounds of the invention may also be admixed, encapsulated, conjugated or otherwise associated with other molecules, molecule structures or mixtures of compounds, as for example, liposomes, receptor targeted molecules, oral, rectal, topical or other formulations, for assisting in uptake, distribution and/or absorption. Representative United States patents that teach the preparation of such uptake, distribution and/or absorption assisting formulations include, but are not limited to, U.S. Pat. Nos. 5,108,921; 5,354,844; 5,416,016; 5,459,127; 5,521,291; 5,543,158; 5,547,932; 5,583,020; 5,591,721; 4,426,330; 4,534,899; 5,013,556; 5,108,921; 5,213,804; 5,227,170; 5,264,221; 5,356,633; 5,395,619; 5,416,016; 5,417,978; 5,462,854; 5,469,854; 5,512,295; 5,527,528; 5,534,259; 5,543,152; 5,556,948; 5,580,575; and 5,595,756, each of which is herein incorporated by reference.

The antisense compounds of the present invention can be utilized for diagnostics, therapeutics, prophylaxis and as research reagents and kits. For therapeutics, an animal, preferably a human, suspected of having tularemia is treated by administering antisense compounds in accordance with this invention. The compounds of the invention can be utilized in pharmaceutical compositions by adding an effective amount of an antisense compound to a suitable pharmaceutically acceptable diluent or carrier. Use of the antisense compounds and methods of the invention may also be useful prophylactically, e.g., to prevent or delay infection, inflammation or tumor formation, for example.

An antisense molecule capable of hybridizing to the nucleic acid under physiologic conditions according to the invention may be used as a probe or as a medicament or may be included in a pharmaceutical composition with a pharmaceutically acceptable carrier, diluent or excipient therefore to treat the particular lymphoma. Nucleic acid molecules according to the invention may be inserted into the vectors described in an antisense orientation in order to provide for the production of antisense RNA. Antisense RNA or other antisense nucleic acids, including antisense peptide nucleic acid (PNA), may be produced by synthetic means.

Some embodiments also include using short interfering RNA to interfere with the expression of the guaA and guaB gene. US Patent Application 20040023390 (the entire contents of which are hereby incorporated by reference as if fully set forth herein) teaches that double-stranded RNA (dsRNA) also called short interfering RNA (siRNA) herein, can induce sequence-specific post-transcriptional gene silencing in many organisms by a process known as RNA interference (RNAi). Recent work suggests that RNA fragments are the sequence-specific mediators of RNAi (Elbashir et al., 2001). Interference of gene expression by these short interfering RNA (siRNA, usually about 19-21 nucleotides long) is now recognized as a naturally occurring strategy for silencing genes in C. elegans, Drosophila, plants, and in mouse embryonic stem cells, oocytes and early embryos (Cogoni et al., 1994; Baulcombe, 1996; Kennerdell, 1998; Timmons, 1998; Waterhouse et al., 1998; Wianny and Zernicka-Goetz, 2000; Yang et al., 2001; Svoboda et al., 2000, the entire contents of which are hereby incorporated by reference as if fully set forth herein).

In mammalian cell culture, a siRNA-mediated reduction in gene expression has been accomplished by transfecting cells with synthetic RNA nucleic acids (Caplan et al., 2001; Elbashir et al., 2001. US patent application publication 20040023390, the entire contents of which are hereby incorporated by reference as if fully set forth herein, provides methods using a viral vector containing an expression cassette containing a pol II promoter operably-linked to a nucleic acid sequence encoding a short interfering RNA molecule (siRNA) targeted against a gene of interest.

As used herein RNAi is the process of RNA interference. A typical mRNA produces approximately 5,000 copies of a protein. RNAi is a process that interferes with or significantly reduces the number of protein copies made by an mRNA. For example, a double-stranded short interfering RNA (siRNA) molecule is engineered to complement and match the protein-encoding nucleotide sequence of the target mRNA to be interfered with using methods known in the art and described herein. Following intracellular delivery, the siRNA molecule associates with a RNA-induced silencing complex (RISC). The siRNA-associated RISC binds the target mRNA through a base-pairing interaction and degrades it. The RISC remains capable of degrading additional copies of the targeted mRNA. Other forms of RNA can be used such as short hairpin RNA and longer RNA molecules. Longer molecules cause cell death, for example by instigating apoptosis and inducing an interferon response. Cell death was the major hurdle to achieving RNAi in mammals because dsRNAs longer than 30 nucleotides activated defense mechanisms that resulted in non-specific degradation of RNA transcripts and a general shutdown of the host cell. Using from about 20 to about 29 nucleotide siRNAs to mediate gene-specific suppression in mammalian cells has apparently overcome this obstacle. These siRNAs are long enough to cause gene suppression but not of a length that induces an interferon response.

Pharmaceutical Compositions Comprising Antisense Molecules

The present invention also includes pharmaceutical compositions and formulations which include the antisense compounds of the invention and a pharmaceutically acceptable carrier or diluent. The pharmaceutical compositions comprising antisense compounds of the present invention may be administered in a number of ways depending upon whether local or systemic treatment is desired and upon the area to be treated. Administration may be topical (including ophthalmic and to mucous membranes including vaginal and rectal delivery), pulmonary, e.g., by inhalation or insufflation of powders or aerosols, including by nebulizer; intratracheal, intranasal, epidermal and transdermal), oral or parenteral. Parenteral administration includes intravenous, intraarterial, subcutaneous, intraperitoneal or intramuscular injection or infusion; or intracranial, e.g., intrathecal or intraventricular, administration. Nucleic acids with at least one 2′-O-methoxyethyl modification are believed to be particularly useful for oral administration.

While any suitable carrier known to those of ordinary skill in the art may be employed in the pharmaceutical compositions of this invention, the type of carrier will typically vary depending on the mode of administration. Compositions of the present invention may be formulated for any appropriate manner of administration, including for example, topical, oral, nasal, mucosal, intravenous, intracranial, intraperitoneal, subcutaneous and intramuscular administration.

Carriers for use within such pharmaceutical compositions are biocompatible, and may also be biodegradable. In certain embodiments, the formulation preferably provides a relatively constant level of active component release. In other embodiments, however, a more rapid rate of release immediately upon administration may be desired. The formulation of such compositions is well within the level of ordinary skill in the art using known techniques. Illustrative carriers useful in this regard include microparticles of poly(lactide-co-glycolide), polyacrylate, latex, starch, cellulose, destran and the like. Other illustrative delayed-release carriers include supramolecular biovectors, which comprise a non-liquid hydrophilic core (e.g., a cross-linked polysaccharide or oligosaccharide) and, optionally, an external layer comprising an amphiphilic compound, such as a phospholipid (see e.g., U.S. Pat. No. 5,151,254 and PCT applications WO94/20078, WO/94/23701 and WO96/06638). The amount of active compound contained within a sustained release formulation depends upon the site of implantation, the rate and expected duration of release and the nature of the condition to be treated or prevented.

Methods of Treatment

The present invention is further directed to methods for treating tularemia in an animal. In one embodiment, the present invention encompasses methods for treating tularemia in an animal comprising blocking expression of the guaA gene of F. tularensis by administering to an animal in need of treatment a therapeutically effective amount of an antisense oligonucleotide that is sufficiently complementary to the guaA gene of F. tularensis to permit specific hybridization under physiologic conditions and wherein the antisense nucleic acid inhibits expression of the guaA gene, thereby treating tularemia in an animal. In a similar embodiment, the present invention encompasses methods for treating tularemia in an animal, comprising blocking expression of the guaB gene of F. tularensis by administering to an animal in need of treatment a therapeutically effective amount of an antisense oligonucleotide that is sufficiently complementary to the guaB gene of F. tularensis to permit specific hybridization under physiologic conditions and wherein the antisense nucleic acid inhibits expression of the guaB gene, thereby treating tularemia in an animal.

In a further embodiment, the present invention encompasses methods for treating tularemia in an animal, comprising blocking translation of messenger RNA produced from the guaA gene of F. tularensis by administering to an animal in need of treatment a therapeutically effective amount of an antisense oligonucleotide that is sufficiently complementary to messenger RNA produced from the guaA gene to permit specific hybridization under physiologic conditions to the messenger RNA, and wherein the antisense nucleic acid inhibits translation of the messenger RNA, thereby treating tularemia in an animal. Similarly, the present invention encompasses methods for treating tularemia in an animal, comprising blocking translation of messenger RNA produced from the guaB gene of F. tularensis by administering to an animal in need of treatment a therapeutically effective amount of an antisense oligonucleotide that is sufficiently complementary to messenger RNA produced from the guaB gene to permit specific hybridization under physiologic conditions to the messenger RNA, and wherein the antisense nucleic acid inhibits translation of the messenger RNA, thereby treating tularemia in an animal.

In yet a further embodiment, the present invention encompasses methods for preventing tularemia in an animal, comprising blocking translation of messenger RNA produced from the guaA gene of F. tularensis by administering to an animal in need of prevention a therapeutically effective amount of an antisense oligonucleotide that is sufficiently complementary to messenger RNA produced from the guaA gene to permit specific hybridization under physiologic conditions to the messenger RNA, and wherein the antisense nucleic acid inhibits translation of the messenger RNA, thereby preventing tularemia in an animal. Similarly, the present invention encompasses methods for preventing tularemia in an animal, comprising blocking translation of messenger RNA produced from the guaB gene of F. tularensis by administering to an animal in need of prevention a therapeutically effective amount of an antisense oligonucleotide that is sufficiently complementary to messenger RNA produced from the guaB gene to permit specific hybridization under physiologic conditions to the messenger RNA, and wherein the antisense nucleic acid inhibits translation of the messenger RNA, thereby preventing tularemia in an animal.

Polynucleotides

Moreover, the present invention is directed to isolated polynucleotide molecules including the isolated polynucleotide molecules comprising the F. tularensis guaA gene polynucleotide sequence set forth in SEQ ID NO:1, and the F. tularensis guaB gene polynucleotide sequence set forth in SEQ ID NO:2.

In a related embodiment, the present invention is directed to an isolated polynucleotide molecule comprising a polynucleotide sequence having at least about 50%, 55%, 60%, 65%, 70%, 75%, 80%, 85%, 90%, 91%, 92%, 93%, 94%, 95%, 96%, 97%, 98% or 99% identitiy to the polynucleotide set forth in SEQ ID NO:1. Similarly, the present invention is directed to an isolated polynucleotide molecule comprising a polynucleotide sequence having at least about 50%, 55%, 60%, 65%, 70%, 75%, 80%, 85%, 90%, 91%, 92%, 93%, 94%, 95%, 96%, 97%, 98% or 99% identity to the polynucleotide set forth in SEQ ID NO:2.

In a further embodiment, the present invention is directed to an isolated polynucleotide molecule comprising a polynucleotide sequence that hybridizes under stringent hybridization conditions to a complement of the polynucleotide set forth in SEQ ID NO:1. Similarly, the present invention is directed to an isolated polynucleotide molecule comprising a polynucleotide sequence that hybridizes under stringent hybridization conditions to a complement of the polynucleotide set forth in SEQ ID NO:1.

As used herein, stringent hybridization conditions mean hybridization in a solution comprising 50% formamide, 6×SSC, 5× Denhardt's solution, 0.5% SDS and 100 ug/ml denatured, sheared salmon sperm DNA at 42° C., followed by filter washing in 0.1×SSC at about 65° C.

In the foregoing specification, the invention has been described with reference to specific embodiments thereof. It will, however, be evident that various modifications and changes may be made thereto without departing from the broader spirit and scope of the invention. The specification and drawings are, accordingly, to be regarded in an illustrative rather than a restrictive sense.

All publications and materials referenced herein, including patents, patent application publications, journal articles, books, manuals, abstracts, and other scientific publications, are each expressly incorporated herein in their entireties.

### Examples

### Example 1

Generation of the guaA and guaB Mutants

The suicide plasmids were created by ligating fragments from various other plasmids required for all functionality. It is known that oriE1 plasmids do not replicate in F. tularensis which therefore allows oriE1 to serve as a suicide plasmid in Ft. It has been reported that most promoters do not function in F. tularensis. For example, the expression of sacB in the guaA suicide plasmid was driven by the F. tularensis groEL promoter.

The invention is also directed to the F. tularensis (guaA) gene encoding GMP synthetase, which gene is defined by SEQ ID NO:1 (1028643-1030193, accession number AJ749949), and to the F. tularensis gene (guaB) encoding IMP dehydrogenase, which gene is defined by SEQ ID NO:2 (1342806-1344266, accession number AJ749949).

The materials and methods description set forth below is one example of how the deletion mutations can be obtained. Variations on this procedure can be made by those skilled in the art. For example, various flanking regions can be used to generate a deletion in the guaB or guaA gene. In addition, alternative methods for allelic exchange can be used including the lambda red recombinase system or direct transformation with DNA fragments.

A. guaB Suicide Plasmids (pFT611 and pFT724)

The 2,686 by pUC19 plasmid (GenBank Accession: X02514) was cut with SmaI and ligated with the 1,142 by cml fragment, previously generated by PCR from pACYC184 plasmid (GenBank Accession: X06403) with the P439 and P442 primers (all relevant primers are shown in Table 1 below). The 3,828 by plasmid generated was named pUC19Cml.

The sacB gene was obtained by digestion of pBS-SacB plasmid with EcoRV (This plasmid previously generated in our laboratory harbors the sacB gene under its native promoter). The ˜1,910 by sacB fragment was inserted in pUC19Cml plasmid as an EcoRV fragment, generating a 5,738 by pUC19CmlSacB plasmid.

To generate a suicide plasmid containing the deleted guaB-encoding region, the flanking upstream and downstream regions were amplified by PCR, using Francisella tularensis LVS chromosomal DNA as a template (the LVS strain was kindly donated by Dr. Karen L Elkins, CBER/FDA, Rockville, Md.). 500 by from the upstream encoding guaB sequence were amplified by PCR with primers P445 and P466. Similarly, 500 by from the encoding downstream guaB sequence were amplified by PCR using the P475 and P446 primers. Both fragments were cloned in a pBluescript plasmid (Genbank Accession: X52328). This plasmid contains the amp-resistance marker and the ColE1 origin.

The chromosomal upstream and downstream fragments in the pBluescript plasmid were separated by the kanamycin (aphT) resistance marker sequence, which was obtained by amplifying a 1,439 by PCR fragment containing the aphT gene from the plasmid pKD4 (GenBank Accession: AY048743) with primers P461 and P462. The 5400 by plasmid generated was named pBSguaB(km).

The 2,440 by guaB(km) cassette was removed from pBSguaB(km) as a SpeI-SalI fragment and inserted in pUC19CmlSacB, previously digested with XbaI-SalI. The new ˜7840 by plasmid generated was named pFT611.

In order to generate the guaB suicide plasmid for Francisella tularensis Schu S4, the cml and amp markers were removed from pFT611. The Cml marker was removed by cutting pFT611 with BamHI, the derived plasmid was named pUC19SacBguaB(Km). Then, the amp marker was removed by inverse PCR with P697 and P698 primers. The PCR product (lacking of the amp marker) was religated and transformed into E. coli. Several colonies were screened by ampicillin sensitivity on LB plates. The derived plasmid was named pFT724.

Plasmid pFT611 and pFT724 were purified according to the Qiagen specifications and used to electroporate the LVS strain.

TABLE 1

| | | SEQ | |
|---|---|---|---|
| | | ID | |
| Primer | Sequence | NO: | Notes |
| P439 | ATCCCCGGGGATATCGTAAGTTGGCAGCATCACCCGAC | 3 | Binds upstream of cml(r) |
| | | | marker |
| P442 | GCCCATCTCTAGAAACTGGCCTCAGGCATTTGAGAA | 4 | Binds downstream of |
| | | | cml(r) marker |
| P443 | CCCGGATCCATCTTTAGAGATAAGTTTTCACATATTGAG | 5 | Binds upstream of |
| | | | flanking guaA region (5′) |
| P444 | CCCGTCGACGCGGCCGCAGATCTATACCGCCAGCATGAT | 6 | Binds upstream of |
| | TTAAGGCTTCA | | flanking guaA region (5′) |
| P445 | CCCGGATCCTAAATATAAGCCTAAGGCAGTGATTGATTT | 7 | Binds upstream of |
| | | | flanking guaB region (5′) |
| P446 | CCCGTCGACGCGGCCGCAACAACACCGCTTACAGCAAA | 8 | Binds downstream of |
| | CTTTTTA | | flanking guaB region (3′) |
| P457 | GATGATATCGCTATGTCAAAATATACTATTTTAGATAAA | 9 | Binds in the beginning of |
| | ATAA | | downstream flanking |
| | | | guaA region (3′) |
| P458 | GATGATATCTTTTTATAAAAATAAACATTTTCTTAAAAG | 10 | Binds in the end of |
| | GTTATTT | | upstream of flanking |
| | | | guaA region (5′) |
| P461 | ATGCCCGGGGAAGTTCCTATACTTTCTAGAGAATAG | 11 | Binds upstream of km (r) |
| | | | marker region (5′) |
| P462 | ATGCCCGGGAGTTCCTATTCCGAAGTTCCTATTCT | 12 | Binds downstream of |
| | | | km(r) marker region (3′) |
| P466 | GATCCCCGAATTCCTGCAGCCCGGGTTTTTGATCTCCGT | 13 | Binds in the end of |
| | AATTAAAATCTAAAGAGT | | upstream flanking guaB |
| | | | region (5′) |
| P475 | CCCGAATTCCTGCAGAACCTCTTAATTATGATTTTAATA | 14 | Binds in the beginning of |
| | AGTTATAAT | | downstream flanking |
| | | | guaB region (3′) |
| P528 | GGATAAAATAACCTTTTAAGAAAATGTTT | 15 | Binds upstream of |
| | | | flanking guaA region (5′) |
| P530 | ACTCTTTAGATTTTAATTACGGAGATC | 16 | Binds upstream of |
| | | | flanking guaB region (5′) |
| P533 | ATG CAG CCG CCG CAT TGC ATC A | 17 | Binds to the km marker |
| P534 | TCTTTTTAATCGCTCCTTGAGAAGC | 18 | Binds to the native guaB |
| | | | encoding region |
| P535 | GTTTCTGGGTGAAACTGCACACC | 19 | Binds to the native guaA |
| | | | encoding region |
| P580 | GAATTCCGGATCCTTTCTTGAAAATTTTTTTTTTGACTCA | 20 | Binds upstream of groEL |
| | ATAT | | promoter (5′) |
| P665 | GAATTCCCCCGGGCTTACTCCTTTGTTAAATTATTTTTGT | 21 | Binds downstream of |
| | TTA | | groEL promoter (3′) |
| P666 | GAATTCCCCCGGGATGAACATCAAAAAGTTTGCAAAAC | 22 | Binds upstream of sacB |
| | AA | | encoding region (5′) |
| P696 | CTAGCTAGGATCCGATATCAAGCTGGGGTGGGCGAAGA | 23 | Binds downstream of |
| | ACT | | km(r) marker (3′) |
| P697 | GCTAGATAGGTGCCTCACTGATTAAGCATT | 24 | Binds downstream of |
| | | | pUC19 plasmid (3′) |
| P698 | ATCGCTAGGATCCGCGAAAGGGGGATGTGCTGCAAG | 25 | Binds upstream of |
| | | | pUC19 plasmid (5′) |
| P878 | TAAATATAAGCCTAAGGCAGTGATTGATTT | 26 | F. tularensis Schu S4 |
| | | | unmarked guaB deletion |
| | | | primer |
| P879 | TTTTGATCTCCGTAATTAAAATCTAAAGAGT | 27 | F. tularensis Schu S4 |
| | | | unmarked guaB deletion |
| | | | primer |
| P880 | TATTCTTATGAATAATACCAATACCACCTT | 28 | F. tularensis Schu S4 |
| | | | unmarked guaB deletion |
| | | | primer |
| P960 | CCCGGGATGGATCCGTCGACACCACGTGGATAACGTAC | 29 | Binds downstream of |
| | CATTGCTGGACCATTAT | | flanking guaA region (3′) |
| P961 | CCCGGGATGGATCCGCCTTACTGACATTACTTGTAGCAT | 30 | Binds upstream of |
| | TATGTTTTGCTG | | flanking guaA region (5′) |
| P966 | GTCGTTACAGATCTGACATCAGAATCATAAACTGACTCA | 31 | Binds in the end of |
| | GGACCACCTG | | upstream flanking guaA |
| | | | region (5′) |
| P967 | GTCGTTACAGATCTAGAACCATTAAGAGAGCTTTTCAAA | 32 | Binds in the beginning of |
| | GATGAGGTTCGT | | downstream flanking |
| | | | guaA region (3′) |
| P1015 | GCTAGTCCTGATATCGGATCCTTTTTTGATCTCCGTAATT | 33 | Binds downstream of |
| | AAAATCTAAAGAGTAT | | groEL promoter (3′) |
| P1016 | GCTAGTCCTAGATCTAAGCTTGTAAATTCTAATACTTAT | 34 | Binds upstream of groEL |
| | TTCTGGCATATTTTTTC | | promoter (5′′) |
| P1049 | ATGACAGATATACATAATCATAAGAT | 35 | F. tularensis Schu S4 |
| | | | unmarked guaA deletion |
| | | | primer |
| P1050 | CCACAGAGTATCACATCCGCAG | 36 | F. tularensis Schu S4 |
| | | | unmarked guaA deletion |
| | | | primer |
| P1051 | GGGCTATCTGGAGGTGTTGAT | 37 | F. tularensis Schu S4 |
| | | | unmarked guaA deletion |
| | | | primer |
| P1052 | TGATCACCAACTACACCTACCG | 38 | F. tularensis Schu S4 |
| | | | unmarked guaA deletion |
| | | | primer |
| P1053 | CTGCGGATGTGATACTCTGTGG | 39 | F. tularensis Schu S4 |
| | | | unmarked guaA deletion |
| | | | primer |
| P1054 | GATACCCCTTAGGGCATCTAAG | 40 | F. tularensis Schu S4 |
| | | | unmarked guaA deletion |
| | | | primer |
| PRD1A | TTTATATAGGTAAATGTTTTACCTGTACCA | 41 | Francisella primer |
| PRD1B | GCCGAGTTTGATGCTGAAAA | 42 | Francisella primer |

B. guaA Suicide Plasmids (pFT695)

A 2671 by DNA fragment containing the guaA gene was amplified by PCR from F. tularensis LVS genomic DNA with P443 and P444 primers. The PCR product comprised the guaA encoding sequence, as well as 500 by from each genomic guaA flanking sequences. The PCR fragment was inserted into a pUC19 plasmid previously digested with SmaI. The 5357 by plasmid generated was named pUC19guaA. The guaA-encoding sequence from pUC19guaA was deleted by PCR with P457 and P458 primers, and replaced by km marker as a SmaI fragment. The new plasmid generated was named pUC19guaA(km).

A new version of pUC19CmlSacB (step2) was generated. This version contains the cml marker under control of the groEL promoter (the cml promoter was not functional in F.t). The plasmid was named: pUC19P_{groEL}CmlSacB. The guaA(km) fragment was cloned in pUC19P_{groEL}CmlSacB as a BamHI fragment. The ˜8037 by plasmid generated was named pUC19P_{groEL}CmlSacBguaA(km).

The pUC19P_{groEL}CmlSacBguaA(km) plasmid was electroporated in F. tularensis. Several cointegrant colonies were generated and verified by PCR. However, after curing the plasmid from the strain with sucrose, no mutants were generated (the native sacB promoter was not functional in F.t). The plasmid pUC19P_{groEL}CmlSacBguaA(km) was improved by removing the cml marker by PCR and replacing the sacB promoter for groEL promoter (primers P665 and P666). The latest plasmid was named: pFT695.

pFT695 plasmid was purified and used to generate LVS ΔguaA mutant. To generate Schu S4 mutants in guaA, the pFT758 plasmid was constructed by moving the km marker to a region outside of the guaA flanking regions, and by removing the amp marker by PCR.

C. Francisella tularensis Electroporation

Suicide plasmids were purified according to the Qiagen specifications. Plasmid generated from 100 ml of bacteria culture was used for each F. tularensis LVS eletroporation. To prepare electrocompetent LVS cells, the strain was grown on Mueller-Hinton 10% blood plates (MHA-B). The bacteria was pelleted in sucrose 0.5 M solution (wash solution) and washed out 4 times with this wash solution at room temperature. The electrocompetent cells were mixed with the suicide plasmid and electroporated (1.75 KV, 25 μF, 600Ω). The bacteria suspension was subjected at three serial pulses. The electroporated suspension was placed into 1.5 ml of MHB, incubated with shaking for 3 h at 37° C., and plated on MHA-B with 10 μg ml⁻¹ of km. Isolated colonies were analyzed by PCR to evaluate the suicide plasmid integration in the F. tularensis genome.

### D. PCR Confirmation of Cointegration

Isolated colonies were observed on MHA-B plates 5 to 7 days after electroporation. Genomic DNA was isolated from colonies according to the Genome®DNA KIT (Q-BIOgene, Carlsbad, Calif.) manufacturer's protocol and used as PCR template. The primers used for screening of guaB cointegrants were: P530, P533 and P534, while that for guaA were: P528, P533, P535. The PCR fragments as well as the expected sizes are described in Table 2.

TABLE 2

| | | GuaB Cointegrant | |
|---|---|---|---|
| | Parental | (LVS::pFT611 or | GuaA Cointegrant |
| Primers | LVS | LVS::pFT724) | (LVS::pFT695) |
| P530, P533, and P534 | P530-P534 (550 bp) | P530-P533 (840 bp) | P530-P534 (550 bp) |
| | | P530-P534 (550 bp) | |
| P528, P533, and P535 | P528-P535 (565 bp) | P528-P535 (565 bp) | P528-P533 (855 bp) |
| | | | P528-P535 (565 bp) |
| PRD1a and PRD1b | 924 bp | 924 bp | 924 bp |

A positive cointegrant colony was selected and grown overnight in Mueller-Hinton Broth complemented with 10 μl/ml of kanamycin and 1 mg of guanine, when the bacteria suspension was approximately 0.4 at 600 nm, the bacterial was spread on MHA-B plates.

### E. Selection of Mutants

Isolated colonies were screened for the guanine auxotrophic phenotype on MHA-B plates with or without guanine The guanine auxotrophic colonies were confirmed by PCR. The primers used for guaB mutants were: P530, P533 and P534, while that for guaA were: P528, P533, P535. The PCR fragments as well as the expected sizes are described in Table 3.

TABLE 3

| | Parental | Mutant | Mutant |
|---|---|---|---|
| Primers | LVS | (LVS ΔguaB) | (LVS ΔguaA) |
| P530, P533, and P534 | P530-P534 (550 bp) | P530-P533 (840 bp) | P530-P534 (550 bp) |
| P528, P533, and P535 | P528-P535 (565 bp) | P528-P535 (565 bp) | P528-P533 (855 bp) |
| PRD1a and PRD1b | 924 bp | 924 bp | 924 bp |

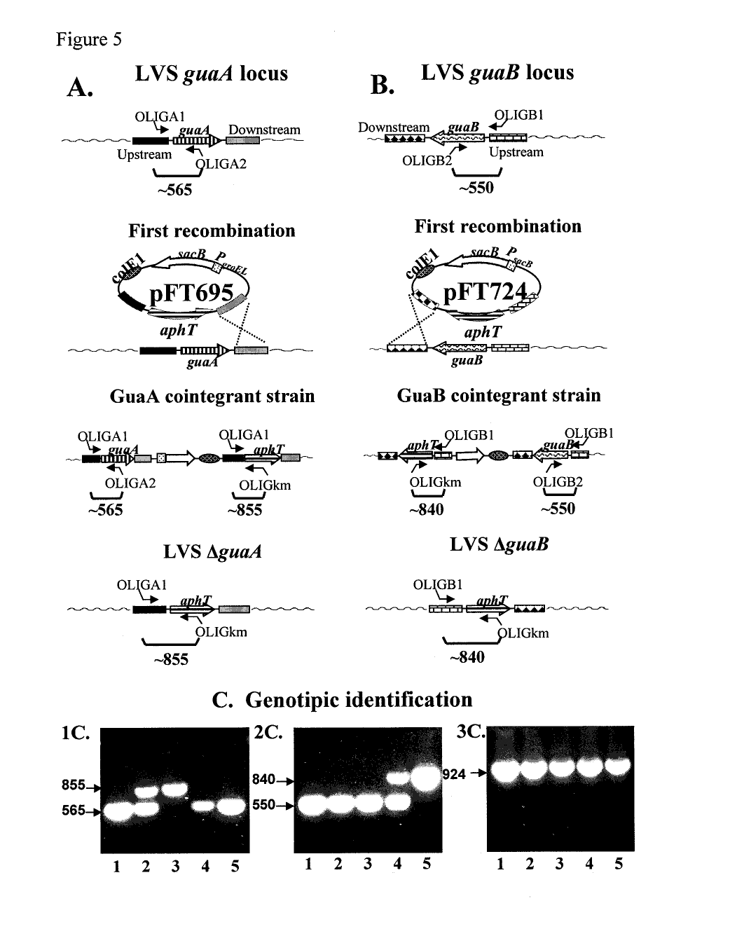

FIG. 5 is a schematic diagram showing the use of allelic exchange technology to create ΔguaA and ΔguaB mutations. The DNA sequence for the guaA (SEQ ID NO:1) and guaB (SEQ ID NO:2) genes are set forth in the Sequence Listing. The guaA and guaB deletions were confirmed by polymerase chain reaction (PCR). FIG. 5 shows the results of genotype analysis of ΔguaB (panel B and 2C) and ΔguaA mutants (panel A and 1C) by PCR amplified fragments using electrophoresis. A set of three specific primers for each gene were used: the guaB locus (Panel 2C: P530, P533 and P534), and the guaA locus (Panel 1C: P528, P533 and P535). For F. tularensis the specific primers (Panel 3C) PRD1A and PRD1B were used.

F. guaB Suicide Plasmids for Schu S4 (pFT840)

In order to generate Schu S4 ΔguaB mutant without km antibiotic resistance marker inside the deleted region, the plasmid pFT840 was created. Plasmid pFT705 was digested with SmaI and the kanamycin resistance marker was removed from the middle of guaB flanking region. The derived plasmid was named pFT711.

At same time, plasmid pFT748 was constructed by cloning the km marker under PgroEl promoter regulation. By using plasmid pFT748 as template, the km marker including the groEL promoter was amplified by PCR (primers P696 and P580) and cloned in plasmid pFT711 as BamHI fragment. The new plasmid was named pFT819.

The amp resistance marker was removed from pFT819 by inverse PCR with P697 and P698 primers. The PCR product (lacking of the amp marker) was ligated and transformed into E. coli. Several colonies were screened by ampicillin sensitivity on LB plates. The derived plasmid was named pFT840.

Plasmid pFT840 was successfully used to delete guaB region in Schu S4 strain. We generated a universal plasmid to delete genes in Schu S4, this plasmid is derived from plasmid pFT840. GuaB region was removed by PCR and replaced by a multiple cloning sites. The plasmid generated was named pFT849.

G. guaA Suicide Plasmids for Schu S4 (pFT894)

In order to generate Schu S4 ΔguaA mutant with no antibiotic markers in the genome, the plasmid pFT894 was constructed. Plasmid pFT894 has 1500 by each end to recombine in the Francisella genome, and it also contains a truncated guaA gene region (890 by were deleted from 1550 by of guaA gene).

Plasmid FT863 was constructed by cloning a fragment of 4550 by of guaA region (this fragment includes 1500 by of each end of open reading frame of guaA gene) in plasmid pFT849 as a BamHI fragment. The guaA region was amplified by PCR by using primer P960 and P961. The plasmid pFT863 contains intact guaA region.

In the plasmid pFT863, a partial encoding guaA region was deleted by inverse PCR (the primers used for this propose were P966, P967). The newest plasmid was named pFT885.

To optimize the functionality of SacB enzyme in Francisella, a second promoter was inserted upstream of sacB gene region. The guaB promoter was amplified by PCR (P1015, P1016) and it was digested with BglII/EcoRV and inserted in plasmid pFT885, already digested with BamHI/EcoRV. The derived plasmid was named pFT894.

Plasmid pFT894 was evaluated in Schu S4. Four guaA mutants were generated. The strains generated are auxotrophic for guanine in vitro. The mutants were evaluated by PCR.

H. Francisella tularensis Electroporation

Suicide plasmids were purified according to the Qiagen specifications. Plasmid generated from 100 ml of bacteria culture was used for each Schu S4 eletroporation. To prepare electrocompetent cells, the strain was grown on Mueller-Hinton 10% blood plates (MHA-B). The bacteria was pelleted in sucrose 0.5 M solution (wash solution) and washed out 4 times with this wash solution at room temperature. The electrocompetent cells were mixed with the suicide plasmid and electroporated (1.75 KV, 25 μF, 600Ω). The bacteria suspension was subjected at three serial pulses. The electroporated suspension was placed into 1.5 ml of MHB, incubated with shaking for 3 h at 37° C., and plated on MHA-B with 10 μg ml⁻¹ of km. Isolated colonies were analyzed by PCR to evaluate the suicide plasmid integration in the F. tularensis genome.

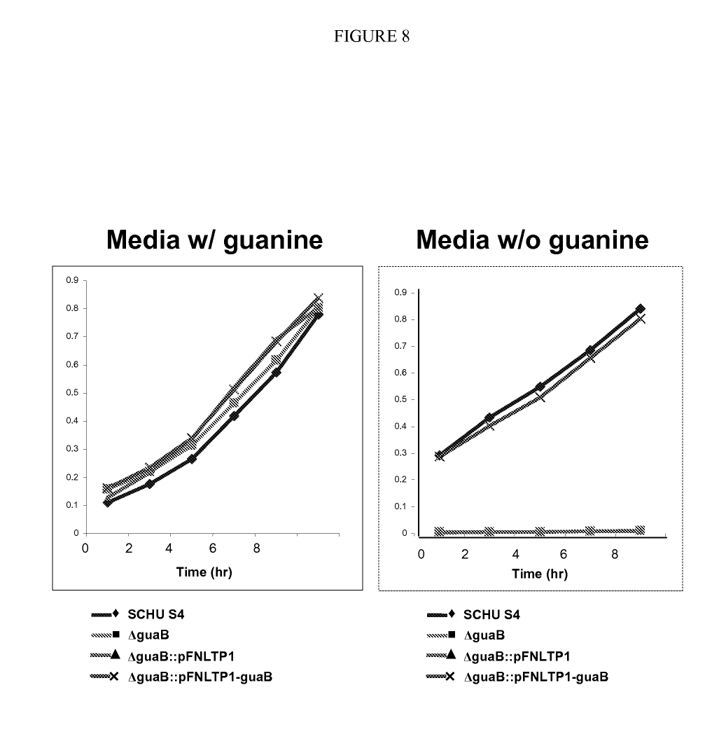

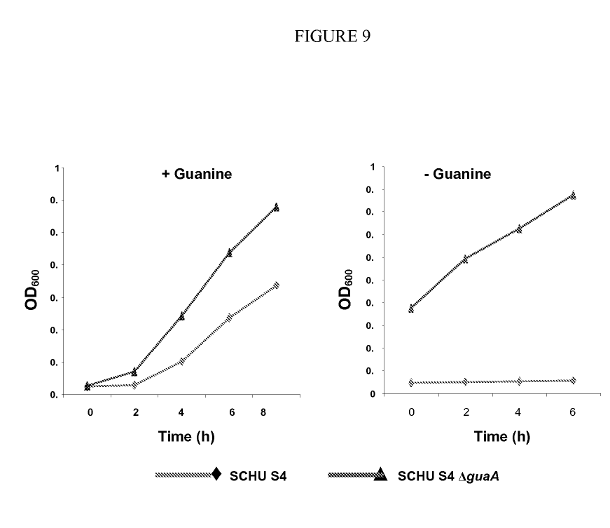

Plasmid pFT840 was used to create an unmarked guaB deletion in Schu S4. Plasmid pFT894 was used to create the unmarked guaA mutation in Schu S4. The derivatives were confirmed for guanine auxotrophy in growth curves (FIGS. 8 and 9). A complementation plasmid was engineered based on Francisella replicon pFNLTP1 with the removal of the amp resistance gene and insertion of the guaB gene to create pFNLT1-guaB. This plasmid was introduced into the guaB deleted Schu S4 derivative and growth in media without guanine was restored (FIG. 8).

### I. PCR Confirmation of Cointegration

Isolated colonies were observed on MHA-B plates 5 to 7 days after electroporation. Genomic DNA was isolated from colonies according to the Genome®DNA KIT (Q-BIOgene, Carlsbad, Calif.) manufacturer's protocol and used as PCR template. Schu S4 ΔguaB(km) cointegrants were: P530, P533 and P534 (Table 4), while those for ΔguaA(km) mutants were: P528, P533, and P535 (Table 5). The PCR fragments as well as the expected sizes are described in Tables 4 and 5.

When Schu S4 ΔguaB mutant without km marker in the genome was generated, different sets of primers were used for screening. Such Schu S4 ΔguaB mutants were analyzed by using primers P530, P534, P878, P879, P880. The PCR fragments as well as the expected sizes are described in Table 6. Similar strategy was used for Schu S4 guaA mutants lacking the km marker, the clones were analyzed by PCR by using primers: P1049, P1050, P1051, P1052, P1053, P1054 (Table 7).

### J. Curing Cointegrants

A positive cointegrant colony was selected and grown overnight in Mueller-Hinton Broth complemented with 10 μl/ml of kanamycin and 1 mg of guanine, when the bacteria suspension was approximately 0.4 at 600 nm, the bacterial was spread on MHA-B plates.

### K. Selection of Mutants

Isolated colonies were screened for the guanine auxotrophic phenotype on MHA-B plates with or without guanine The guanine auxotrophic colonies were confirmed by PCR. The primers used for guaB mutants were: P530, P533 and P534 (see Table 4), while that for guaA were: P528, P533, P535 (see Table 5). Schu S4 ΔguaB mutant was analyzed by using primers: P530, P534, P878, P879, P880 (see Table 6). Schu S4 ΔguaA mutant was evaluated by using primers: P1049, P1050, P1051, P1052, P1053, P1054 (see Table 7).

TABLE 4
guaB mutant (including Km resistance marker
inside of the deleted guaB region)

| | Parental | | Mutant |
|---|---|---|---|
| Primers | F. tularensis | GuaB Cointegrant | (F. tularensis ΔguaB) |
| P530, P533, P534 | P530-P534 (550 bp) | P530-P533 (840 bp) | P530-P533 (840 bp) |
| | | P530-P534 (550 bp) | |
| PRD1a PRD1b | 924 bp | 924 bp | 924 bp |

TABLE 5
guaA mutant (including Km resistance marker inside of the deleted guaA region)

| | Parental | | Mutant |
|---|---|---|---|
| Primers | F. tularensis | GuaA Cointegrant | (F. tularensis ΔguaA) |
| P528, P533, P535 | P528-P535 (565 bp) | P528-P533 (855 bp) | P528-P533 (855 bp) |
| | | P528-P535 (565 bp) | |
| PRD1a PRD1b | 924 bp | 924 bp | 924bp |

TABLE 6
Sizes of resultant PCR products using Schu S4 ΔguaB mutant
(without km marker) as a template compared to wild-type

| | Wild type | | ΔguaB mutant |
|---|---|---|---|
| Primers | Schu S4 | ΔguaB mutant | complemented |
| P878, P880 | 720 bp | negative | negative |
| P878, P879 | 500 bp | 500 bp | 500 bp |
| P530, P534 | 500 bp | negative | 500 bp |

TABLE 7
Sizes of resultant PCR products using Schu S4 ΔguaA mutant
(without km marker) as a template compared to wild-type

| | Wild type | | ΔguaA mutant |
|---|---|---|---|
| Primers | Schu S4 | ΔguaA mutant | complemented |
| P1050, P1049 | 600 bp | negative | 600 bp |
| P1053, P1054 | 305 bp | negative | 305 bp |
| P1049, P1052 | 1360 bp | 480 bp | 1360 bp, 480 bp |
| P1051, P1052 | 700 bp | neg | 700 bp |

### Example 2

Growth of the guaA and guaB Mutants In Vitro

The auxotrophic guanine phenotypes of F. tularensis ΔguaA and ΔguaB were evaluated in vitro. Isolated F. tularensis derivatives were inoculated in 5 ml of MHB media supplemented with guanine and incubated overnight at 37° C. After that, 100 μl of overnight inoculum was placed into 50 ml of fresh supplemented guanine media and incubated with shaking at 37° C. A second bacteria culture lacking of guanine was prepared following the procedure previously indicated. Both inoculated bacteria cultures were incubated with shaking at 37° C. and the optical density at 600 nm was evaluated at 600 nm every 2 h.

### Example 3

Intracellular Growth of the SCHU S4 guaB Mutant

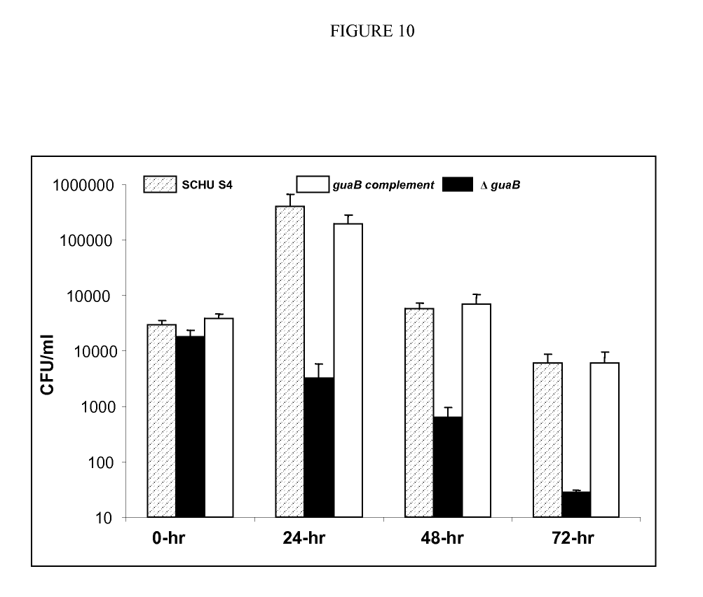

The intracellular multiplication of Schu S4ΔguaB mutants was evaluated in macrophages J774 (American Type Culture Collection, Manassas, Va.) versus the wild-type Schu S4 strain and Schu S4ΔguaB complement with pFNLTP1-guaB. The cell line was cultivated in Dulbecco's modified essential medium (DMEM) (Cellgro® Herndon, Va.), supplemented with 2 mM glutamine (Gibco, Grand Island, N.Y.) and 10% heat-inactivated defined fetal bovine serum (Gibco). Infections were performed in 12-well plates (Costar, Corning, N.Y.). 3×10⁵ cell per well were infected at a multiplicity of infection (MOI) of 100 for 2 h and maintained at 37° C. in humidified air containing 5% CO₂. Following this period, cells were washed three times with PBS, and incubated in DMEM medium with 50 μg/ml of gentamicine (Gibco) for an hour. The cells were washed and incubated in DMEM medium with 2 μg/ml⁻¹ of gentamicine. Replication in macrophages was evaluated at 0, 24, 48 and 72 h post-infection by lysis of cells with SDS 0.02%-PBS solution and plating of 10-fold dilutions on MHA-B plates (FIG. 10). The guaB deleted SchuS4 derivative failed to replicate following infection. When this strain was complemented with pFNLTP1-guaB, wild type levels of replication were restored. Time zero in the assay was evaluated in pre-treated cells with 50 μg ml⁻¹ of gentamicin.

### Example 4

### Growth of the SCHU S4 guaB Mutant In Vivo

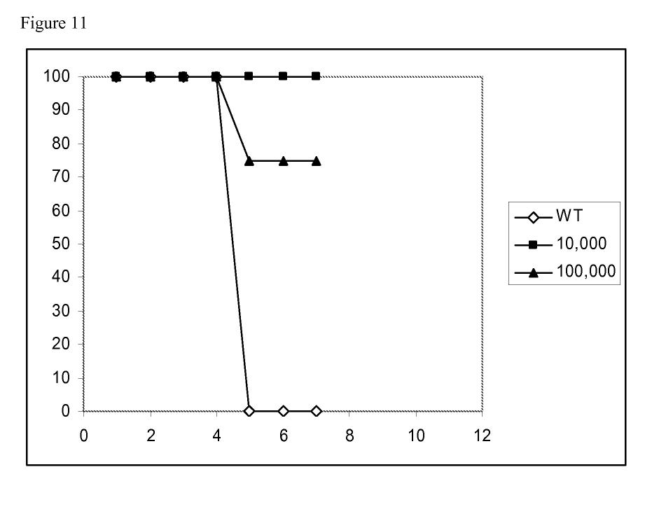

A mouse model was used to evaluate the SchuS4 guaB deleted mutant for attenuation of virulence (FIG. 11). Four mice per group were inoculated by the intranasal route with the following inocula. Wild type Schu S4 at 400 CFU; guaB deleted Schu S4 at 10,000 CFU; or guaB attenuated Schu S4 at 100,000 CFU. 100% of the wild type infected mice were dead by day 5, whereas, the guaB mutant did not kill the mice by Day 7 at a 25-fold higher dose, and only killed 25% of the mice at a 250-fold higher dose by Day 7.

### Example 5

Growth of the guaA AND guaB Mutants In Vivo

Studies were performed to assess the survival and dissemination of the mutant strains in vivo in the mouse. Groups of 9 mice each were inoculated with the parental LVS or the guaA or guaB mutant strain. 24, 48 and 72 hours post inoculation, 3 mice from each group were euthanized and organs harvested for colony counts (Table 8). The parental LVS-inoculated mice showed progressive increases in bacterial counts in the spleen, lung, liver and blood reaching bacterial burdens of 10⁶, 10⁴, 10⁷ CFU in spleen, lung and liver respectively by 72 hours. No bacteria were detected in any organ in the guaA or guaB mutant inoculated mice at any time point following inoculation except for 10 CFU detected in the spleen of 2/3 mice inoculated with the guaB mutant at 24-hours. These studies confirmed the attenuation of virulence of the mutant strains in vivo.

In particular, groups of 3 to 4 BALB/c mice were challenged with LVS derivatives in 1 ml of gelatin 1%-PBS. Groups 1, 4 and 7 were inoculated with 100 CFU of F. tularensis LVS ΔguaA; Groups 2, 5 and 8 received 720 CFU of LVS ΔguaB and groups 3, 6 and 9 were inoculated with 860 CFU of parental LVS by the intraperitoneal route. Mice were sacrificed on day 1 (groups: 1, 2, and 3), day 2 (groups: 4, 5 and 6) and day 3 of infection (groups: 7, 8 and 9 ). Tissue homogenates were serially diluted in PBS and plated on MHA-B plates, and the number of CFU per organ was calculated.

TABLE 8
Growth of F. tularensis in organs of infected naïve BALB/c mice.

| Group | F. tularensis | Mean ± SD CFU of Francisella (log₁₀)/organ | | | |
|---|---|---|---|---|---|
| (No. mice) | strain^{a} | Spleen | Lung | Liver | Blood (ml) |
| 1 (n = 3) | LVS ΔguaA | BDL^{b} | BDL | BDL | BDL |
| 2 (n = 3) | LVS ΔguaB | 1.03 ± 0.91 (2/3)^{c} | BDL | BDL | BDL |
| 3 (n = 3) | LVS | 4.31 ± 0.76 | 2.82 ± 1.18 | 4.48 ± 0.91 | 2.17 ± 0.96 |
| 4 (n = 3) | LVS ΔguaA | BDL | BDL | BDL | BDL |
| 5 (n = 3) | LVS ΔguaB | BDL | BDL | BDL | BDL |
| 6 (n = 4) | LVS | 5.91 ± 0.78 | 3.82 ± 1.69 | 5.87 ± 0.90 | >2.00^{d} |
| 7 (n = 3) | LVS ΔguaA | BDL | BDL | BDL | BDL |
| 8 (n = 4) | LVS ΔguaB | BDL | BDL | BDL | BDL |
| 9 (n = 4) | LVS | 6.77 ± 0.95 | 4.20 ± 0.95 | 6.92 ± 1.09 | >2.00 |

^{b}BDL, below detection limit (~20 organisms/organ)
^{c}Bacteria only detected in two of three organs
^{d}Bacteria detected in the organs is over 100 CFU/ml of blood

Table 9 illustrates the attenuation of virulence of the guaA and guaB mutant strains in the mouse model of inoculation compared to the parental LVS strain. Mice were inoculated i.p. with the indicated doses of the LVS (Groups 1-5), derivative strains (LVS ΔguaA: Groups 6-11; LVS ΔguaB: Groups 12-17) or control (Group 18). Survival was recorded for 28 days. Survival ratio=number of mice alive/total number of mice injected with the corresponding LVS derivative at day 28. Mice inoculated with LVS at a dose of 1.7×10² CFU were all dead by 8 days post inoculation. In contrast, 5/5 of the mice inoculated with the guaA strain alive following a dose of 9.0×10⁶ CFU and 4/5 were alive following a dose of 9.0×10⁷ CFU. All mice (5/5) inoculated with a dose of 1.0×10⁷CFU of the guaB mutant strain were alive 28 days following inoculation.

TABLE 9

| | Bacterial | | |
|---|---|---|---|
| Experimental | inoculum | Survival | Time to death of |
| group^{a} | (CFU) | ratio^{b} | individual mice (days) |
| LVS | | | |
| Group 1 | 1.7 × 10¹ | 3/5 | 5, 7, >28, >28, >28 |
| Group 2 | 1.7 × 10² | 0/5 | 5, 5, 6, 6, 8 |
| Group 3 | 1.7 × 10³ | 0/5 | 4, 6, 6, 6, 7 |
| Group 4 | 1.7 × 10⁴ | 0/5 | 3, 3, 4, 5, 5 |
| Group 5 | 1.7 × 10⁵ | 0/5 | 3, 3, 3, 4, 6 |
| LVS ΔguaA | | | |
| Group 6 | 9.0 × 10² | 5/5 | >28, >28, >28, >28, >28 |
| Group 7 | 9.0 × 10³ | 5/5 | >28, >28, >28, >28, >28 |
| Group 8 | 9.0 × 10⁴ | 5/5 | >28, >28, >28, >28, >28 |
| Group 9 | 9.0 × 10⁵ | 5/5 | >28, >28, >28, >28, >28 |
| Group 10 | 9.0 × 10⁶ | 5/5 | >28, >28, >28, >28, >28 |
| Group 11 | 9.0 × 10⁷ | 4/5 | 3, >28, >28, >28, >28 |
| LVS ΔguaB | | | |
| Group 12 | 1.05 × 10² | 5/5 | >28, >28, >28, >28, >28 |
| Group 13 | 1.05 × 10³ | 5/5 | >28, >28, >28, >28, >28 |
| Group 14 | 1.05 × 10⁴ | 5/5 | >28, >28, >28, >28, >28 |
| Group 15 | 1.05 × 10⁵ | 5/5 | >28, >28, >28, >28, >28 |
| Group 16 | 1.05 × 10⁶ | 5/5 | >28, >28, >28, >28, >28 |
| Group 17 | 1.05 × 10⁷ | 5/5 | >28, >28, >28, >28, >28 |
| Gelatin 0.1%-PBS | | | |
| control group | | | |
| Group 18 | none | 5/5 | >28, >28, >28, >28, >28 |

### Example 6

Protective Effects of guaA AND guaB Mutants In Vivo

Challenge studies in mice indicated that vaccination with either mutant strain protected mice from subsequent lethal challenge with the parental LVS strain (Table 10). Groups of mice received a single immunization by the i.p. route with the ΔguaA or ΔguaB mutant strain at a dose of 10², 10⁴, 10⁶, or 10⁷ CFU. The results show a remarkable efficacy of vaccination with the deletion mutants. 100% of the mice in all groups that received either the ΔguaA or ΔguaB strain at a dose of 10⁴ CFU or higher were protected against a lethal challenge with 10⁴ CFU of LVS. By contrast, none of the control animals immunized with PBS survived the challenge with LVS.

Groups of BALB/c mice were vaccinated (see Table 9) with 10 fold dilutions of LVS ΔguaA (groups 6 to 11); LVS ΔguaB (groups 12 to 17) and gelatin 0.1%—PBS (group 18) The animals were challenged with 2.3×10³ CFU of LVS by intraperitoneal route and survival was observed by 28 days. Survival ratio, number of survival animal/total number of animals immunized at day 28.

TABLE 10
Protective immunity in vaccinated BALB/c mice
to challenge with F. tularensis LVS

| | Immunization | Survival | Median time to death |
|---|---|---|---|
| Vaccine Strain | dose | ratio^{c} | (days) |
| Experiment No. 1^{a} | | | |
| LVS ΔguaA | | | |
| Group 6 | 9.0 × 10² | 4/5 | >28 |
| Group 8 | 9.0 × 10⁴ | 5/5 | >28 |
| Group 10 | 9.0 × 10⁶ | 5/5 | >28 |
| Group 11 | 9.0 × 10⁷ | 4/4^{d} | >28 |
| LVS ΔguaB | | | |
| Group 12 | 1.05 × 10² | 0/5 | 5 |
| Group 14 | 1.05 × 10⁴ | 5/5 | >28 |
| Group 16 | 1.05 × 10⁶ | 5/5 | >28 |
| Group 17 | 1.05 × 10⁷ | 5/5 | >28 |
| Gelatin 0.1%-PBS | | | |
| Group 18 | | | |

A second challenge experiment (Table 11) utilizing a higher challenge dose of 2.8×10⁵ CFU parental LVS administered intraperitoneally confirmed the protective capacity of a single immunization with 2.2×10⁷ LVS ΔguaA (group 1) or 3.6×10⁷ LVS ΔguaB (group 2) mutant strain. Survival ratio, number of survival animal/total number of animals immunized at day 28.

One mouse died after the immunization with the LVS guaB-mutant strain.

TABLE 11

| Vaccine | Immunization | Survival | Median time to death |
|---|---|---|---|
| Strain | dose | ratio^{c} | (days) |
| LVS ΔguaA | | | |
| Group 1 | 2.2 × 10⁷ | 10/10 | >28 |
| LVS ΔguaB | | | |
| Group 2 | 3.6 × 10⁷ | 9/9^{d} | >28 |
| Gelatin 0.1%-PBS | | | |
| Group 3 | None | 0/10 | 5 |

### REFERENCES

1. Petersen, J. M. and M. E. Schriefer. 2005. Tularemia: emergence/re-emergence. Vet. Res. 36:455-467.
2. Elkins, K. L., S. C. Cowley, and C. M. Collazo. 2003. Francisella: a little bug hits the big time. Expert. Rev. Vaccines. 2:735-738.
3. Sjostedt, A. 2003. Virulence determinants and protective antigens of Francisella tularensis. Curr. Opin. Microbiol. 6:66-71.
4. Tarnvik, A. 1989. Nature of protective immunity to Francisella tularensis. Rev. Infect. Dis. 11:440-451.
5. 2002. Tularemia—United States, 1990-2000. MMWR Morb. Mortal. Wkly. Rep. 51:181-184.
6. Oyston, P. C., A. Sjostedt, and R. W. Titball. 2004. Tularaemia: bioterrorism defence renews interest in Francisella tularensis. Nat. Rev. Microbiol. 2:967-978.
7. Kortepeter, M. G. and G. W. Parker. 1999. Potential biological weapons threats. Emerg. Infect. Dis. 5:523-527.
8. Elkins, K. L., S. C. Cowley, and C. M. Bosio. 2003 Innate and adaptive immune responses to an intracellular bacterium, Francisella tularensis live vaccine strain. Microbes. Infect. 5:135-142.
9. Tarnvik, A., M. Eriksson, G. Sandstrom, and A. Sjostedt. 1992. Francisella tularensis—a model for studies of the immune response to intracellular bacteria in man. Immunology 76:349-354.
10. Eigelsbach, H. T. and C. M. DOWNS. 1961. Prophylactic effectiveness of live and killed tularemia vaccines. I. Production of vaccine and evaluation in the white mouse and guinea pig. J. Immunol. 87:415-425.
11. SASLAW, S., H. T. EIGELSBACH, H. E. WILSON, J. A. PRIOR, and S. CARHART. 1961. Tularemia vaccine study. I. Intracutaneous challenge. Arch. Intern. Med. 107:689-701.
12. SASLAW, S., H. T. EIGELSBACH, J. A. PRIOR, H. E. WILSON, and S. CARHART. 1961. Tularemia vaccine study. II. Respiratory challenge. Arch. Intern. Med. 107:702-714.
13. DIENST, F. T., Jr. 1963. Tularemia: a perusal of three hundred thirty-nine cases. J. La State Med. Soc. 115:114-127.
14. Elkins, K. L., R. K. Winegar, C. A. Nacy, and A. H. Fortier. 1992. Introduction of Francisella tularensis at skin sites induces resistance to infection and generation of protective immunity. Microb. Pathog. 13:417-421.
15. Fortier, A. H., M. V. Slayter, R. Ziemba, M. S. Meltzer, and C. A. Nacy. 1991. Live vaccine strain of Francisella tularensis: infection and immunity in mice. Infect. Immun. 59:2922-2928.
16. Green, M., G. Choules, D. Rogers, and R. W. Titball. 2005. Efficacy of the live attenuated Francisella tularensis vaccine (LVS) in a murine model of disease. Vaccine 23:2680-2686.
17. Elkins, K. L., T. Rhinehart-Jones, C. A. Nacy, R. K. Winegar, and A. H. Fortier. 1993. T-cell-independent resistance to infection and generation of immunity to Francisella tularensis. Infect. Immun. 61:823-829.
18. Elkins, K. L., D. A. Leiby, R. K. Winegar, C. A. Nacy, and A. H. Fortier. 1992. Rapid generation of specific protective immunity to Francisella tularensis. Infect. Immun. 60:4571-4577.

What is claimed is:

1. A strain of Francisella tularensis that cannot form de novo guanine nucleotides, wherein said strain has a non-functional guaA gene.

2. A strain of F. tularensis that cannot form de novo guanine nucleotides, wherein said strain has a non-functional guaB gene.

3. A strain of F. tularensis that cannot form de novo guanine nucleotides, wherein said strain has a non-functional guaA gene and has a non-functional guaB gene.

4. The strain of claim 1 or 3, wherein said non-functional guaA gene has one or more of a deletion, insertion, substitution or rearrangement.

5. The strain of claim 2 or 3, wherein said non-functional guaB gene has one or more of a deletion, insertion, substitution or rearrangement.

6. The strain of claim 1, 2 or 3, wherein said strain is a recombinant strain.

7. A vaccine formulation comprising:
(a) a pharmaceutically effective amount of a strain of F. tularensis that cannot form de novo guanine nucleotides, wherein said strain has a non-functional guaA gene, or a non-functional guaB gene, or both a non-functional guaA gene and has a non-functional guaB gene, and
(b) a pharmaceutically acceptable carrier or diluent.

8. The vaccine formulation of claim 7, wherein the pharmaceutically effective amount is about 10 to about 1×10¹¹ cfu/ml.

9. A pFT724 guaB suicide plasmid.

10. A pFT695 guaA suicide plasmid.

11. A pFT758 guaA suicide plasmid.

12. The suicide plasmid of claim 9, 10 or 11, further comprising one or more selectable markers selected from the group consisting of kanamycin (km), chloramphenicol, tetracycline, and ampicillin.

13. The suicide plasmid of claim 9, 10 or 11, further comprising a promoter operably linked to sacB.

14. The suicide plasmid of claim 13, wherein the promoter is a native promoter of sacB or groEL.

15. A method of generating an immune response in a subject comprising administering an immunologically effective amount of a vaccine of claim 7 to a subject, thereby generating an immune response in a subject.

16. A method of generating a protective immune response in a subject comprising administering an immunologically effective amount of a vaccine of claim 7 to a subject, thereby generating an immune response in a subject.

17. A method for treating tularemia in an animal, comprising blocking expression of the guaA gene of F. tularensis by administering to an animal in need of treatment a therapeutically effective amount of an antisense oligonucleotide that is sufficiently complementary to the guaA gene of F. tularensis to permit specific hybridization under physiologic conditions and wherein said antisense nucleic acid inhibits expression of the guaA gene, thereby treating tularemia in an animal.

18. A method for treating tularemia in an animal, comprising blocking expression of the guaB gene of F. tularensis by administering to an animal in need of treatment a therapeutically effective amount of an antisense oligonucleotide that is sufficiently complementary to the guaB gene of F. tularensis to permit specific hybridization under physiologic conditions and wherein said antisense nucleic acid inhibits expression of the guaB gene, thereby treating tularemia in an animal.

19. A method for treating tularemia in an animal, comprising blocking translation of messenger RNA produced from the guaA gene of F. tularensis by administering to an animal in need of treatment a therapeutically effective amount of an antisense oligonucleotide that is sufficiently complementary to messenger RNA produced from the guaA gene to permit specific hybridization under physiologic conditions to the messenger RNA, and wherein said antisense nucleic acid inhibits translation of the messenger RNA, thereby treating tularemia in an animal.

20. A method for preventing tularemia in an animal, comprising blocking translation of messenger RNA produced from the guaA gene of F. tularensis by administering to an animal in need of prevention a therapeutically effective amount of an antisense oligonucleotide that is sufficiently complementary to messenger RNA produced from the guaA gene to permit specific hybridization under physiologic conditions to the messenger RNA, and wherein said antisense nucleic acid inhibits translation of the messenger RNA, thereby preventing tularemia in an animal.

21. A method for treating tularemia in an animal, comprising blocking translation of messenger RNA produced from the guaB gene of F. tularensis by administering to an animal in need of treatment a therapeutically effective amount of an antisense oligonucleotide that is sufficiently complementary to messenger RNA produced from the guaB gene to permit specific hybridization under physiologic conditions to the messenger RNA, and wherein said antisense nucleic acid inhibits translation of the messenger RNA, thereby treating tularemia in an animal.

22. A method for preventing tularemia in an animal, comprising blocking translation of messenger RNA produced from the guaB gene of F. tularensis by administering to an animal in need of prevention a therapeutically effective amount of an antisense oligonucleotide that is sufficiently complementary to messenger RNA produced from the guaB gene to permit specific hybridization under physiologic conditions to the messenger RNA, and wherein said antisense nucleic acid inhibits translation of the messenger RNA, thereby preventing tularemia in an animal.

23. An isolated polynucleotide molecule comprising a polynucleotide molecule selected from the group consisting of:
(i) the F. tularensis guaA gene polynucleotide sequence set forth in SEQ ID NO:1,
(ii) an isolated polynucleotide molecule comprising a polynucleotide sequence having at least 95% identity to the polynucleotide set forth in SEQ ID NO:1, and
(iii) an isolated polynucleotide molecule comprising a polynucleotide sequence that hybridizes under stringent hybridization conditions to a complement of the polynucleotide set forth in SEQ ID NO:1.

24. An isolated polynucleotide molecule comprising a polynucleotide molecule selected from the group consisting of:
(i) the F. tularensis guaB gene polynucleotide sequence set forth in SEQ ID NO:2,
(ii) an isolated polynucleotide molecule comprising a polynucleotide sequence having at least 95% identity to the polynucleotide set forth in SEQ ID NO:2, and
(iii) an isolated polynucleotide molecule comprising a polynucleotide sequence that hybridizes under stringent hybridization conditions to a complement of the polynucleotide set forth in SEQ ID NO:2.
